SEQUENCE LISTING (37 sequences)

SEQ ID NO: 1 - DNA, 833 bp, Homo sapiens
  CDS (1)...(810)
agaaaatacccactttctcaggatgatatcaatggaatccagtccatctatggaggtctgcctaaggaacctgctaagccaaaggaacccactataccccatgcctgtgaccctgacttgacttttgacgctatcacaactttccgcagagaagtaatgttctttaaaggcaggcacctatggaggatctattatgatatcacggatgttgagtttgaattaattgcttcattctggccatctctgccagctgatctgcaagctgcatacgagaaccccagagataagattctggtttttaaagatgaaaacttctggatgatcagaggatatgctgtcttgccagattatcccaaatccatccatacattaggttttccaggacgtgtgaagaaaatagatgcagccgtctgtgataagaccacaagaaaaacctacttctttgtgggcatttggtgctggaggtttgatgaaatgacccaaaccatggacaaagggttcccgcagagagtggtaaaacactttcctggaatcagtatccgtgttgatgctgctttccagtacaaaggattcttctttttcagccgtggatcaacgcaatttgaatacgacattaagacaaagaatattacccgaatcatgagaactaatacttggtttcaatgcaaagaaccaaagaactcctcatttggttttgatatcaacaaggaaaaagcacattcaggaggcataaagatattgtatcataagagtttaagcttgtttatttttggtattgttcatttgctgaaaaacacttctatttatcaataaattcatagacctaaaataaa

SEQ ID NO: 2 - protein, 269 aa, Homo sapiens
KYPLSQDDINGIQSIYGGLPKEPAKPKEPTIPHACDPDLTFDAITTFRREVMFFKGRHLWRIYYDITDVEFELIASFWPSLPADLQAAYENPRDKILVFKDENFWMIRGYAVLPDYPKSIHTLGFPGRVKKIDAAVCDKTTRKTYFFVGIWCWRFDEMTQTMDKGFPQRVVKHFPGISIRVDAAFQYKGFFFFSRGSTQFEYDIKTKNITRIMRTNTWFQCKEPKNSSFGFDINKEKAHSGGIKILYHKSLSLFIFGIVHLLKNTSIYQ

SEQ ID NO: 3 - DNA, 1488 bp, Homo sapien
ggcttactcactatagggctcgagcggccgcccgggcaggtgaaagagaggaatgaagcgccttctgcttctgtttttgttctttataacattttcttctgcatttcccttagtccggatgatggaaaatgaagaaaatgtgcaactggctcaggcatatctcaaccagttctactctcttgaaatagaagggaatcatcttgttcaaagcaagaataggagtctcatagatgacaaaattcgggaaatgcaagcattttttggattgacagtgactggaagactggactcaaacacccttgagatcatgaagacacccaggtgtggggtgcctgatgtgggccagtatggctacaccctccctgggtggagaaaatacaacctcacctacagaataataaactatactccggatatggcacgagctgctgtggatgaggctatccaagaaggtttagaagtgtggagcaaagtcactccactaaaattcaccaagatttcaaaggggattgcagacatcatgattgcctttaggactcgaggattcaacttgtttcttgtggctgctcatgaatttggtcatgcactggggctctctcactccaatgatcaaacagccttgatgttcccaaattatgtctccctggatcccagaaaatacccactttctcaggatgatatcaatggaatccagtccatctatggaggtctgcctaaggaacctgctaagccaaaggaacccactataccccatgcctgtgaccctgacttgacttttgacgctatcacaactttccgcagagaagtaatgttctttaaaggcaggcacctatggaggatctattatgatatcacggatgttgagtttgaattaattgcttcattctggccatctctgccagctgatctgcaagctgcatacgagaaccccagagataagattctggtttttaaagatgaaaacttctggatgatcagaggatatgctgtcttgccagattatcccaaatccatccatacattaggttttccaggacgtgtgaagaaaatagatgcagccgtctgtgataagaccacaagaaaaacctacttctttgtgggcatttggtgctggaggtttgatgaaatgacccaaaccatggacaaagggttcccgcagagagtggtaaaacactttcctggaatcagtatccgtgttgatgctgctttccagtacaaaggattcttctttttcagccgtggatcaacgcaatttgaatacgacattaagacaaagaatattacccgaatcatgagaactaatacttggtttcaatgcaaagaaccaaagaactcctcatttggttttgatatcaacaaggaaaaagcacattcaggaggcataaagatattgtatcataagagtttaagcttgtttatttttggtattgttcatttgctgaaaaacacttctatttatcaataaattcatagacctaaaataaaaaa

SEQ ID NO: 4 - protein, 466 aa, Homo sapien
MKRLLLLFLFFITFSSAFPLVRMMENEENVQLAQAYLNQFYSLEIEGNHLVQSKNRSLIDDKIREMQAFFGLTVTGRLDSNTLEIMKTPRCGVPDVGQYGYTLPGWRKYNLTYRIINYTPDMARAAVDEAIQEGLEVWSKVTPLKFTKISKGIADIMIAFRTRGFNLFLVAAHEFGHALGLSHSNDQTALMFPNYVSLDPRKYPLSQDDINGIQSIYGGLPKEPKPKEPTIPHACDPDLTFDAITTFRREVMFFKGRHLWRIYYDITDVEFELIASFWPSLPDLQAAYENPRDKILVFKDENFWMIRGYAVLPDYPKSIHTLGFPGRVKKIDAAVCDKTTRKTYFFVGIWCWRFDEMTQTMDKGFPQRVVKHFPGISIRVDAAFQYKGFFFFRGSTQFEYDIKTKNITRIMRTNTWFQCKEPKNSSFGFDINKEKAHSGGIKILYHKSSLFIFGIVHLLKNTSIYQ

SEQ ID NO: 5 - DNA, 1841 bp, Homo sapien
gaaagagaggaatgaagcgccttctgcttctgtttttgttctttataacattttcttctgcatttcccttagtccggatgatggaaaatgaagaaaatgtgcaactggctcaggcatatctcaaccagttctactctcttgaaatagaagggaatcatcttgttcaaagcaagaataggagtctcatagatgacaaaattcgggaaatgcaagcattttttggattgacagtgactggaagactggactcaaacacccttgagatcatgaagacacccaggtgtggggtgcctgatgtgggccagtatggctacaccctccctgggtggagaaaatacaacctcacctacagaataataaactatactccggatatggcacgagctgctgtggatgaggctatccaagaaggtttagaagtgtggagcaaagtcactccactaaaattcaccaagatttcaaaggggattgcagacatcatgattgcctttaggactcgagtccatggtcggtgtcctcgctattttgatggtcccttgggagttcttggccatgcctttcctcctggtccgggtctgggtggtgacactcattttgatgaggatgaaaactggaccaaggatggagcaggattcaacttgtttcttgtggctgctcatgaatttggtcatgcactggggctctctcactccaatgatcaaacagccttgatgttcccaaattatgtctccctggatcccagaaaatacccactttctcaggatgatatcaatggaatccagtccatctatggaggtctgcctaaggaacctgctaagccaaaggaacccactataccccatgcctgtgaccctgacttgacttttgacgctatcacaactttccgcagagaagtaatgttctttaaaggcaggcacctatggaggatctattatgatatcacggatgttgagtttgaattaattgcttcattctggccatctctgccagctgatctgcaagctgcatacgagaaccccagagataagattctggtttttaaagatgaaaacttctggatgatcagaggatatgctgtcttgccagattatcccaaatccatccatacattaggttttccaggacgtgtgaagaaaatagatgcagccgtctgtgataagaccacaagaaaaacctacttctttgtgggcatttggtgctggaggtttgatgaaatgacccaaaccatggacaaagggttcccgcagagagtggtaaaacactttcctggaatcagtatccgtgttgatgctgctttccagtacaaaggattcttctttttcagccgtggatcaacgcaatttgaatacgacattaagacaaagaatattacccgaatcatgagaactaatacttggtttcaatgcaaagaaccaaagaactcctcatttggttttgatatcaacaaggaaaaagcacattcaggaggcataaagatattgtatcataagagtttaagcttgtttatttttggtattgttcatttgctgaaaaacacttctatttatcaataaattcatagacctaaaataaacctcaacaggtcttttaatataaattctgcttcaaaatagaataaaaccattctttaacaacaagttgctggtcctagttctaaatatccaaattcaatggccattttgagctgcctgattcttttaataggaagttattatgtagaaacaaaaatctctgactgtactttaagcctatttcatgctttgtggacttggagaagacatgtcttataactgaatactgaaacatttattaaaccaatctttagcattctgaaaaaaaaa

SEQ ID NO: 6 - protein, 513 aa, Homo sapien
MKRLLLLFLFFITFSSAFPLVRMMENEENVQLAQAYLNQFYSLEIEGNHLVQSKNRSLIDDKIREMQAFFGLTVTGRLDSNTLEIMKTPRCGVPDVGQYGYTLPGWRKYNLTYRIINYTPDMARAAVDEAIQEGLEVWSKVTPLKFTKISKGIADIMIAFRTRVHGRCPRYFDGPLGVLGHAFPPGPGLGGDTHFDEDENWTKDGAGFNLFLVAAHEFGHALGLSHSNDQTALMFPNYVSLDPRKYPLSQDDINGIQSIYGGLPKEPAKPKEPTIPHACDPDLTFDAITTFRREVMFFKGRHLWRIYYDITDVEFELIASFWPSLPADLQAAYENPRDKILVFKDENFWMIRGYAVLPDYPKSIHTLGFPGRVKKIDAAVCDKTTRKTYFFVGIWCWRFDEMTQTMDKGFPQRVVKHFPGISIRVDAAFQYKGFFFFSRGSTQFEYDIKTKNITRIMRTNTWFQCKEPKNSSFGFDINKEKAHSGGIKILYHKSLSLFIFGIVHLLKNTSIYQ

SEQ ID NO: 7 - protein, 27 aa, Homo sapien
  VARIANT (1)...(27): Xaa = any amino acid
LVAAHELGHXLGLXHSXXXXAXMSSSY

SEQ ID NO: 8 - protein, 37 aa, Homo sapiens
  VARIANT (1)...(37): Xaa = any amino acid
HGDXXPFDGXXXXLAHAFXPGXGXGGDXHPDXDEXWT

SEQ ID NO: 9 - DNA, 30 bp, Artificial Sequence
  Primer
tgatatcataatagatcctccataggtgcc

SEQ ID NO: 10 - DNA, 31 bp, Artificial Sequence
  Primer
ttccttaggcagacctccatagatggactgg

SEQ ID NO: 11 - DNA, 27 bp, Artificial Sequence
  Primer
cctaaggaacctgctaagccaaaggaa

SEQ ID NO: 12 - DNA, 25 bp, Artificial Sequence
  Primer
ccgcagagaagtaatgttctttaaa

SEQ ID NO: 13 - DNA, 25 bp, Artificial Sequence
  Primer
ccgcagagaagtaatgttctttaaa

SEQ ID NO: 14 - DNA, 30 bp, Artificial Sequence; identical to SEQ ID NO: 9
  Primer

SEQ ID NO: 15 - DNA, 411 bp, Homo sapien
agaaaatacccactttctcaggatgatatcaatggaatccagtccatctatggaggtctgcctaaggaacctgctaagccaaaggaacccactataccccatgcctgtgaccctgacttgacttttgacgctatcacaactttccgcagagaagtaatgttctttaaaggcaggcacctatggaggatctattatgatatcacggatgttgagtttgaattaattgcttcattctggccatctctgccagctgatctgcaagctgcatacgagaaccccagagataagattctggtttttaaagatgaaaacttctggatgatcagaggatatgctgtcttgccagattatcccaaatccatccatacattaggttttccaggacgtgtgaagaaaatagatgcagccgtc

SEQ ID NO: 16 - DNA, 382 bp, Homo sapiens
tttttttttttattttaggtctatgaatttattgataaatagaagtgtttttcagcaaatgaacaataccaaaaataaacaagcttaaactcttatgatacaatatctttatgcctcctgaatgtgctttttccttgttgatatcaaaaccaaatgaggagttctttggttctttgcattgaaaccaagtattagttctcatgattcgggtaatattctttgtcttaatgtcgtattcaaattgcgttgatccacggctgaaaaagaagaatcctttgtactggaaagcagcatcaacacggatactgattccaggaaagtgttttaccactctctgcgggaaccctttgtccatggtttgggtcatttcatcaaacctcca

SEQ ID NO: 17 - protein, 12 aa, Homo sapien
  VARIANT (3)...(3): Xaa = any amino acid
HEXFHXXGXXHX

SEQ ID NO: 18 - protein, 7 aa, Homo sapiens
  VARIANT (5)...(5): Xaa = any amino acid
PRCGXPD

SEQ ID NO: 19 - protein, 469 aa, Homo sapiens
MHSFPPLLLLLFWGVVSHSFPATLETQEQDVDLVQKYLEKYYNLKNDGRQVEKRRNSGPVVEKLKQMQEFFGLKVTGKPDAETLKVMKQPRCGVPDVAQFVLTEGNPRWEQTHLTYRIENYTPDLPRADVDHAIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN

SEQ ID NO: 20 - protein, 467 aa, Homo sapiens [sequence omitted]

SEQ ID NO: 21 - protein, 471 aa, Homo sapiens
MHPGVLAAFLFLSWTHCRALPLPSGGDEDDLSEEDLQFAERYLRSYYHPTNLAGILKENAASSMTERLREMQSFFGLEVTGKLDDNTLDVMKKPRCGVPDVGEYNVFPRTLKWSKMNLTYRIVNYTPDMTHSEVEKAFKKAFKVWSDVTPLNFTRLHDGIADIMISFGIKEHGDFYPFDGPSGLLAHAFPPGPNYGGDAHFDDDETWTSSSKGYNLFLVAAHEFGHSLGLDHSKDPGALMFPIYTYTGKSHFMLPDDDVQGIQSLYGPGDEDPNPKHPKTPDKCDPSLSLDAITSLRGETMIFKDRFFWRLHPQQVDAELFLTKSFWPELPNRIDAAYEHPSHDLIFIFRGRKFWALNGYDILEGYPKKISELGLPKEVKKISAAVHFEDTGKTLLFSGNQVWRYDDTNHIMDKDYPRLIEEDFPGIGDKVDAVYEKNGYIYFFNGPIQFEYSIWSNRIVRVMPANSILWC

SEQ ID NO: 22 - protein, 267 aa, Homo sapiens
MRLTVLCAVCLLPGSLALPLPQEAGGMSELQWEQAQDYLKRFYLYDSETKNANSLEAKLKEMQKFFGLPITGMLNSRVIEIMQKPRCGVPDVAEYSLFPNSPKWTSKVVTYRIVSYTRDLPHITVDRLVSKALNMWGKEIPLHFRKVVWGTADIMIGFARGAHGDSYPFDGPGNTLAHAFAPGTGLGGDAHFDEDERWTDGSSLGINFLYAATHELGHSLGMGHSSDPNAVMYPTYGNGDPQNFKLSQDDIKGIQKLYGKRSNSRKK

SEQ ID NO: 23 - protein, 470 aa, Homo sapiens
MKFLLILLLQATASGALPLNSSTSLEKNNVLFGERYLEKFYGLEINKLPVTKMKYSGNLMKEKIQEMQHFLGLKVTGQLDTSTLEMMHAPRCGVPDVHHFREMPGGPVWRKHYITYRINNYTPDMNREDVDYAIRKAFQVWSNVTPLKFSKINTGMADILVVFARGAHGDFHAFDGKGGILAHAFGPGSGIGGDAHFDEDEFWTTHSGGTNLFLTAVHEIGHSLGLGHSSDPKAVMFPTYKYVDINTFRLSADDIRGIQSLYGDPKENQRLPNPDNSEPALCDPNLSFDAVTTVGNKIFFFKDRFFWLKVSERPKTSVNLISSLWPTLPSGIEAAYEIEARNQVFLFKDDKYWLISNLRPEPNYPKSIHSFGFPNFVKKIDAAVFNPRFYRTYFFVDNQYWRYDERRQMMDPGYPKLITKNFQGIGPKIDAVFYSKNKYYYFFQGSNQFEYDFLLQRITKTLKSNSWFGC

SEQ ID NO: 24 - protein, 477 aa, Homo sapiens
MKSLPILLLLCVAVCSAYPLDGAARGEDTSMNLVQKYLENYYDLKKDVKQFVRRKDSGPVVKKIREMQKFLGLEVTGKLDSDTLEVMRKPRCGVPDVGHFRTFPGIPKWRKTHLTYRIVNYTPDLPKDAVDSAVEKALKVWEEVTPLTFSRLYEGEADIMISFAVREHGDFYPFDGPGNVLAHAYAPGPGINGDAHFDDDEQWTKDTTGTNLFLVAAHEIGHSLGLFHSANTEALMYPLYHSLTDLTRFRLSQDDINGIQSLYGPPPDSPETPLVPTEPVPPEPGTPANCDPALSFDAVSTLRGEILIFKDRHFWRKSLRKLEPELHLISSFWPSLPSGVDAAYEVTSKDLVFIFKGNQFWAIRGNEVRAGYPRGIHTLGFPPTVRKIDAAISDKEKNKTYFFVEDKYWRFDEKRNSMEPGFPKQIAEDFPGIDSKIDAVFEEFGFFYFFTGSSQLEFDPNAKKVTHTLKSNSWLNC

SEQ ID NO: 25 - protein, 476 aa, Homo sapiens
MMHLAFLVLLCLPVCSAYPLSGAAKEEDSNKDLAQQYLEKYYNLEKDVKQFRRKDSNLIVKKIQGMQKFLGLEVTGKLDTDTLEVMRKPRCGVPDVGHFSSFPGMPKWRKTHLTYRIVNYTPDLPRDAVDSAIEKALKVWEEVTPLTFSRLYEGEADIMISFAVKEHGDFYSFDGPGHSLAHAYPPGPGLYGDIHFDDDEKWTEDASGTNLFLVAAHELGHSLGLFHSANTEALMYPLYNSFTELAQFRLSQDDVNGIQSLYGPPPASTEEPLVPTKSVPSGSEMPAKCDPALSFDAISTLRGEYLFFKDRYFWRRSHWNPEPEFHLISAFWPSLPSYLDAAYEVNSRDTVFIFKGNEFWAIRGNEVQAGYPRGIHTLGFPPTIRKIDAAVSDKEKKKTYFFAADKYWRFDENSQSMEQGFPRLIADDFPGVEPKVDAVLQAFGFFYFFSGSSQFEFDPNARMVTHILKSNSWLHC

SEQ ID NO: 26 - protein, 488 aa, Homo sapiens [sequence omitted]

SEQ ID NO: 27 - protein, 582 aa, Homo sapiens [sequence omitted]

SEQ ID NO: 28 - protein, 669 aa, Homo sapiens
MGSDPSAPGRPGWTGSLLGDREEAARPRLLPLLLVLLGCLGLGVAAEDAEVHAENWLRLYGYLPQPSRHMSTMRSAQILASALAEMQRFYGIPVTGVLDEETKEWMKRPRCGVPDQFGVRVKANLRRRRKRYALTGRKWNNHHLTFSIQNYTEKLGWYHSMEAVRRAFRVWEQATPLVFQEVPYEDIRLRRQKEADIMVLFASGFHGDSSPFDGTGGFLAHAYFPGPGLGGDTHFDADEPWTFSSTDLHGNNLFLVAVHELGHALGLEHSSNPNAIMAPFYQWKDVDNFKLPEDDLRGIQQLYGTPDGQPQPTQPLPTVTPRRPGRPDHRPPRPPQPPPPGGKPERPPKPGPPVQPRATERPDQYGPNICDGDFDTVAMLRGEMFVFKGRWFWRVRHNRVLDNYPMPIGHFWRGLPGDISAAYERQDGRFVFFKGDRYWLFREANLEPGYPQPLTSYGLGIPYDRIDTAIWWEPTGHTFFFQEDRYWRFNEETQRGDPGYPKPISVWQGIPASPKGAFLSNDAAYTYFYKGTKYWKFDNERLRMEPGYPKSILRDFMGCQEHVEPGPRWPDVARPPFNPHGGAEPGADSAEGDVGDGDGDFGAGVNKDGGSRVVVQMEEVARTVNVVMVLVPLLLLLCVLGLTYALVQMQRKGAPRVLLYCKRSLQEWV

SEQ ID NO: 29 - protein, 607 aa, Homo sapiens
MILLTFSTGRRLDFVHHSGVFFLQTLLWILCATVCGTEQYFNVEVWLQKYGYLPPTDPRMSVLRSAETMQSALAAMQQFYGINMTGKVDRNTIDWMKKPRCGVPDQTRGSSKFHIRRKRYALTGQKWQHKHITYSIKNVTPKVGDPETRKAIRRAFDVWQNVTPLTFEEVPYSELENGKRDVDITIIFASGFHGDSSPFDGEGGFLAHAYFPGPGIGGDTHFDSDEPWTLGNPNHDGNDLFLVAVHELGHALGLEHSNDPTAIMAPFYQYMETDNFKLPNDDLQGIQKIYGPPDKIPPPTRPLPTVPPHRSIPPADPRKNDRPKPPRPPTGRPSYPGAKPNICDGNFNTLAILRREMFVFKDQWFWRVRNNRVMDGYPMQITYFWRGLPPSIDAVYENSDGNFVFFKGNKYWVFKDTTLQPGYPHDLITLGSGIPPHGIDSAIWWEDVGKTYFFKGDRYWRYSEEMKTMDPGYPKPITVWKGIPESPQGAFVHKENGFTYFYKGKEYWKFNNQILKVEPGYPRSILKDFMGCDGPTDRVKEGHSPPDDVDIVIKLDNTASTVKAIAIVIPCILALCLLVLVYTVFQFKRKGTPRHILYCKRSMQEWV

SEQ ID NO: 30 - protein, 519 aa, Homo sapiens
MQQFGGLEATGILDEATLALMKTPRCSLPDLPVLTQARRRRQAPAPTKWNKRNLSWRVRTFPRDSPLGHDTVRALMYYALKVWSDIAPLNFHEVAGSTADIQIDFSKADHNDGYPFDGPGGTVAHAFFPGHHHTAGDTHFDDDEAWTFRSSDAHGMDLFAVAVHEFGHAIGLSHVAAAHSIMRPYYQGPVGDPLRYGLPYEDKVRVWQLYGVRESVSPTAQPEEPPLLPEPPDNRSSAPPRKDVPHRCSTHFDAVAQIRGEAFFFKGKYFWRLTRDRHLVSLQPAQMHRFWRGLPLHLDSVDAVYERTSDHKIVFFKGDRYWVFKDNNVEEGYPRPVSDFSLPPGGIDAAFSWAHNDRTYFFKDQLYWRYDDHTRHMDPGYPAQSPLWRGVPSTLDDAMRWSDGASYFFRGQEYWKVLDGELEVAPGYPQSTARDWLVCGDSQADGSVAAGVDAAEGPRAPPGQHDQSRSEDGYEVCSCTSGASSPPGAPGPLVAATMLLLLPPLSPGALWTAAQALTL

SEQ ID NO: 31 - protein, 508 aa, Homo sapiens
MNCQQLWLGFLLPMTVSGRVLGLAEVAPVDYLSQYGYLQKPLEGSNNFKPEDITEALRAFQEASELPVSGQLDDATRARMRQPRCGLEDPFNQKTLKYLLLGRWRKKHLTFRILNLPSTLPPHTARAALRQAFQDWSNVAPLTFQEVQAGAADIRLSFHGRQSSYCSNTFDGPGRVLAHADIPELGSVHFDEDEFWTEGTYRGVNLRIIAAHEVGHALGLGHSRYSQALMAPVYEGYRPHFKLHPDDVAGIQALYGKKSPVIRDEEEEETELPTVPPVPTEPSPMPDPCSSELDAMMLGPRGKTYAFKGDYVWTVSDSGPGPLFRVSALWEGLPGNLDAAVYSPRTQWIHFFKGDKVWRYINFKMSPGFPKKLNRVEPNLDAALYWPLNQKVFLFKGSGYWQWDELARTDFSSYPKPIKGLFTGVPNQPSAAMSWQDGRVYFFKGKVYWRLNQQLRVEKGYPRNISHNWMHCRPRTIDTTPSGGNTTPSGTGITLDTTLSATETTFEY

SEQ ID NO: 32 - protein, 471 aa, Homo sapiens; identical to SEQ ID NO: 21

SEQ ID NO: 33 - protein, 183 aa, Homo sapiens
MDPGTVATMRKPRCSLPDVLGVAGLVRRRRRYALSGSVWKKRTLTWRVRSFPQSSQLSQETVRVLMSYALMAWGMESGLTFHEVDSPQGQEPDILIDFARAFHQDSYPFDGLGGTLAHAFFPGEHPISGDTHFDDEETWTFGSKASQQLEQELAGGSPVDEELGFSRGWRVNPLGPGSPERLS

SEQ ID NO: 34 - protein, 390 aa, Homo sapiens
MGRGARVPSEAPGAGVERRWLGAALVALCLLPALVLLARLGAPAVPAWSAAQGDVAALGLSAVPPTRVPGPLAPRRRRYTLTPARLRWDHFNLTYRILSFPRNLLSPRETRRALAAAFRMWSDVSPFSFREVAPEQPSDLRIGFYPINHTDCLVSALHHCFDGPTGELAHAFFPPHGGIHFDDSEYWVLGPTRYSWKKGVWLTDLVHVAAHEIGHALGLMHSQHGRALMHLNATLRGWKALSQDELWGLHRLYGCLDRLFVCASWARRGFCDARRRLMKRLCPSSCDFCYEFPFPTVATTPPPPRTKTRLVPEGRNVTFRCGQKILHKKGKVYWYKDQEPLEFSYPGYLALGEAHLSIIANAVNEGTYTCVVRRQQRVLTTYSWRVRVRG

SEQ ID NO: 35 - protein, 660 aa, Homo sapiens
MEALMARGALTGPLRALCLLGCLLSHAAAAPSPIIKFPGDVAPKTDKELAVQYLNTFYGCPKESCNLFVLKDTLKKMQKFFGLPQTGDLDQNTIETMRKPRCGNPDVANYNFFPRKPKWDKNQITYRIIGYTPDLDPETVDDAFARAFQVWSDVTPLRFSRIHDGEADIMINFGRWEHGDGYPFDGKDGLLAHAFAPGTGVGGDSHFDDDELWTLGEGQVVRVKYGNADGEYCKFPFLFNGKEYNSCTDTGRSDGFLWCSTTYNFEKDGKYGFCPHEALFTMGGNAEGQPCKFPFRFQGTSYDSCTTEGRTDGYRWCGTTEDYDRDKKYGFCPETAMSTVGGNSEGAPCVFPFTFLGNKYESCTSAGRSDGKMWCATTANYDDDRKWGFCPDQGYSLFLVAAHEFGHAMGLEHSQDPGALMAPIYTYTKNFRLSQDDIKGIQELYGASPDIDLGTGPTPTLGPVTPEICKQDIVFDGIAQIRGEIFFFKDRFIWRTVTPRDKPMGPLLVATFWPELPEKIDAVYEAPQEEKAVFFAGNEYWIYSASTLERGYPKPLTSLGLPPDVQRVDAAFNWSKNKKTYIFAGDKFWRYNEVKKKMDPGFPKLIADAWNAIPDNLDAVVDLQGGGHSYFFKGAYYLKLENQSLKSVKFGSIKSDWLGC

SEQ ID NO: 36 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTRDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEEPLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 37 - protein, 16 aa, Homo sapiens
  VARIANT (3)...(5): Xaa = any amino acid
HGXXXPXFDGXXXHAF

(19) United States
(12) Patent Application Publication
(10) Pub. No.: US 2002/0037827 A1
(43) Pub. Date: Mar. 28, 2002

(54) Novel matrix metalloproteinase (MMP-25) expressed in skin cells

(76) Inventors: Kai Wang, Bellevue, WA (US); Ryan Smith, Seattle, WA (US); Mark Fajardo, Shoreline, WA (US); Patrick Moss, Shoreline, WA (US); Randall C. Schatzman, Shoreline, WA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/801,196

(22) Filed: Mar. 6, 2001

Related U.S. Application Data
(60) Provisional application No. 60/187,196, filed on Mar. 6, 2000.

(51) Int. Cl. A61K 31/00; A01K 67/00; C07H 21/04; C12N 9/64; C12N 5/06; C12P 21/02

(52) U.S. Cl. 514/1; 435/226; 800/8; 435/325; 435/69.1; 536/23.2

(57) ABSTRACT

This invention provides nucleic acids and polypeptides encoding a novel family of matrix metalloproteinases herein designated as MMP-25 and variants of the same. MMP-25 is preferentially expressed in skin cells of a mammal, particularly in breast cells and hair follicles. Expression in hair follicles is localized in the Henle layer of cells, indicating a role in hair growth. Also provided are fragments and oligonucleotides useful for identifying and isolating MMP-25-encoding nucleic acids and methods for their use, as well as antibodies that bind specifically to MMP-25 and vectors for expression of MMP-25 polypeptides. Methods of inhibiting MMP-25 activity are provided, including methods useful for inhibiting hair growth.

### CROSS-REFERENCE TO RELATED APPLICATIONS

This application claims priority to pending U.S. patent application No. 60/187,196 filed Mar. 6, 2000 which is incorporated by reference herein in its entirety.

### TECHNICAL FIELD

This invention relates to matrix metalloproteinases (MMPs), particularly to a class of MMPs herein designated as MMP-25 that is preferentially expressed in skin cells and more particularly, in hair follicles and breast cells. It also relates to polypeptide embodiments of MMP-25, to nucleic acids encoding the same, to antibodies that bind to MMP-25, and to pharmaceutical products and compositions and methods for inhibiting the expression or catalytic activity of MMP-25 sequences.

### BACKGROUND OF THE INVENTION

Matrix metalloproteinases (MMPs) are a family of zinc dependent endopeptidases that function extracellularly to degrade proteins typically found in the extracellular matrix of animal tissue or secreted from bacterial and fungal cells. Members of the MMP family include proteinases designated by common names such as stomelysin or matrilysin, substrate names such as collagenase or gelatinase, and tissue names such as macrophage metalloelastase or neutrophil gelatinase. Alternative nomenclature designates these enzymes by number and include MMP-1 through MMP-22 although the numbering is not sequential. All mammalian tissues are believed to express one or more MMP polypeptides which are exported from the cell or have the catalytic domain located external to the cell in the case of membrane-type matrix metalloproteinases (MT-MMPs) which are anchored to the membrane by a transmembrane domain. Protein substrates for MMPs include collagens, laminins, gelatinins, aggrecans, fibronectins, hyaluronidase treated versican, elastin, cassein, vitronectin, enatactin, fibrin, plasminogen, proteoglycan linked proteins and other MMPs. Most MMPs have overlapping substrate specificity and are able to degrade multiple substrates albeit with different levels of activity.

There at least 22 known family members of zinc dependent MMP that function extracellularly in animal cells. Each of these MMPs contains a first Zn-binding domain that has a conserved HExFHxxGxxHS/T peptide sequence (SEQ ID NO:17) in which three histidine residues form a complex with Zn to form a catalytic protease domain. These MMPs further contain a second Zn-binding domain that is capable of binding calcium, and is sometimes referred to as the Zn/Ca-binding domain. In addition, MMPs contain a regulatory domain pro-peptide toward the N terminus of a pro-protein and which has a conserved PRCGxPD cysteine motif (SEQ ID NO:18) that functions to prevent activation of the pro-protein by binding of the cysteine residue to the active site Zn atom. Activation of the enzyme occurs by proteolytic cleavage of the cysteine motif containing pro-peptide to convert the pro-protein to the active polypeptide. While the catalytic domains of different MMPs have similar structures, differences in other domains of these polypeptides confer substrate specificity and the ability to respond to different regulators such as naturally occurring tissue inhibitors of metalloproteinases (TIMPs) or chemical compounds that inhibit activity.

MMPs are involved in a wide a wide variety of physiological functions related to tissue growth including tissue remodeling and migration of normal and malignant cells in the body. They also serve as regulatory molecules in enzyme cascades by processing a variety of matrix proteins, cytokines, growth factors and adhesion molecules to generate fragments with enhanced or reduced biological effects. As a consequence of their manifold functions related to tissue growth, control of MMP expression from various cell types is an important target for affecting physiological processes as diverse as angiogenesis, hair growth, photoaging of the skin and cancer. For example, Styczynski et al. (U.S. Pat. No. 5,962,466) discloses that inhibition of MMP activity in follicle cells leads to a reduction in hair growth. Voorhees et al. (U.S. Pat. No. 5,837,224) discloses that inhibition of MMP induction in skin cells provides for protection against photoaging of skin. Similarly, De Nanteuil et al. (U.S. Pat. No. 5,866,587) and Docherty et al. (U.S. Pat. No. 5,883,241) each disclose that regulation of MMP is a means to control a variety of growth related pathologies, including breast cancer.

Both direct and indirect inhibition of MMP activity have been described. One form of indirect inhibition involves stimulating an increase in the expression or catalytic activity of a naturally occurring TIMP with compounds such as bromo-cyclic AMP, 3,4 dihydroxybenzaldehyde and estradiol-3-bis(2-chloroethyl)carbamate. Another form of indirect inhibition occurs by increasing the co-expression of a second, inactive form of a MMP in the same tissue as the active enzyme. For example, Rubins et al. (U.S. Pat. No. 5,935,792) discloses that expression of a non-functional variant of KUZ family MMP during neurogenesis of Drosophila cells interferes with the activity of a functional KUZ variant, thereby acting as a dominant negative regulator of MMP activity. Still another form of indirect inhibition is by regulation of transcription factors involved in regulation of cytokine expression such as AP-1 or NF-kappa B, as described for example by Angel et al., Cell 49:729-739 (1987); and Sato and Seiki, Oncogene 8:395-405 (1993). Other transcriptional factors that indirectly regulate MMP expression include those that are responsive to environmental stress such as oxidants, heat or UV irradiation. Devary, Science, 261:1442-1445 (1993); Wlaschek et al., Photochemistry and Photobiology 59:550-556 (1994). These factors are in turn regulated by numerous molecules including for example, RAC, CDC42, MEKK, JNKK, JNK, RAS, RAF, MED AND ERK.

A variety of chemical inhibitors for inhibition of MMP activity have also been described. These include inhibitors of transcriptional factors that regulate MMP expression and inhibitors of the catalytic activity of the polypeptide. Examples include CT1166 and R0317467, Hill et al., Biochem J. 308:167-175 (1995); hydroxamates, thiols, phosphonates, phosphinates, phosphoramidates and n-carboxy alkyls as mentioned by Gowravaram et al., J. Med. Chem. 38:2570-2581 (1995); Galardin, Batimastat and Marimastat, Hodgson, J. Biotechnology 13:554-557 (1995); butanediamide, Conway et al., J. Exp. Med 182:449-457 (1995); retinoids, Fanjul et al., Nature 372:107-111 (1994); Nicholson et al., EMBO Journal 9:4443-4445 (1990), and Bailey et al., J. Investig. Derm. 94:47-51 (1990). In addition, Golub et al. (U.S. Pat. No. 5,837,696) discloses that a variety of chemically modified tetracyclines are effective MMP inhibitors at concentrations below those required for their ordinary purpose of conferring antimicrobial activity.

MMPs encompass a diverse family of enzymes distinguished by different tissue specificity, different substrate specificity and different responsiveness to activators or inhibitors Therefore, there is a need in the art to identity unique MMPs polypeptides, nucleic acids, and genes that encode the same. There is also a need to determine particular patterns of tissue expression and chromosome locations for these novel MMPs so as to provide methods for regulating physiological functions associated with the same. The present invention provides for these needs by identifying a unique sub-family of MMPs nucleic acids and polypeptides particularly expressed in skin tissue, particularly hair follicles and breast cells, which are useful targets for inhibitors for controlling hair growth, breast cancer and other conditions associated with this particular MMP and its variants..

### SUMMARY OF THE INVENTION

The present invention provides sequence for a novel MMP herein designated as MMP-25. More specifically, the invention provides an isolated nucleic acid comprising a nucleotide sequence selected from the group consisting of: (a) a sequence according to SEQ ID NO:1 or SEQ ID NO:3; or SEQ ID NO:5; (b) a sequence that is a complement of (a); and (c) a sequence that hybridizes under conditions of normal stringency to the sequence of (a) or (b). In a similar aspect the invention provides an isolated nucleic acid comprising a nucleotide sequence selected from the group consisting of a sequence encoding a polypeptide according to SEQ ID NO:2, SEQ ID NO:4 or SEQ ID NO:6; a sequence encoding a polypeptide having at least 50% identity to the polypeptide of (a); a sequence encoding a functional fragment of the polypeptide of (a) or (b); and a nucleic acid sequence that is a complement of (a)-(c).

Also provided herein are nucleic acid fragments useful as probes and primers for identifying or obtaining a MMP-25 sequences. In this aspect, the invention provides a nucleic acid fragment or oligonucleotide comprising at least 15 contiguous nucleotides of SEQ ID NO:1 or SEQ ID NO:3, or SEQ ID NO:5 or a compliment thereof, with the proviso that said nucleic acid fragment is not SEQ ID NO:15 or 16. Other embodiments include a nucleic acid fragment or oligonucleotide comprising at least 15 contiguous nucleotides selected from positions 1-653 of SEQ ID NO:3 or a compliment thereof; and a nucleic acid fragment or oligonucleotide comprising at least 15 contiguous nucleotides selected from positions 1-741 or 1573-1841 of SEQ ID NO:5 or a compliment thereof. Particular embodiments of these nucleic acid fragments or oligonucleotides include any of the above where the length is at least 18, 24, 30, 50 or greater than 50 nucleotides.

In a related aspect, the invention provides a nucleic acid fragment or oligonucleotide encoding a peptide comprised of at least 8 contiguous amino acids of the sequence according to SEQ ID NO:2, SEQ ID NO:4 or SEQ ID NO:6, with the proviso that said nucleic acid fragment is not SEQ ID NO:15 or 16. Particular embodiments of this aspect include nucleic acid fragments or oligonucleotides encoding a peptides comprised of at least 10, 15, or 20 amino acids. Still more particular embodiments include the aforementioned nucleic acid fragments wherein the encoded peptide comprises contiguous amino acids from positions 1-61, 98-111, 161-170 or 261-513 of SEQ ID NO:6.

In a similarly related aspect, the invention provides a nucleic acid fragment or oligonucleotide encoding a peptide comprised of at least 8 contiguous amino acids from positions 1-200 of SEQ ID NO:4. Particular embodiments of this aspect also include fragments or oligonucleotides comprised of at least 10, 15 or 20 amino acids. Also included within this aspect are any one of these fragments or oligonucleotides wherein the peptide comprises contiguous amino acids from positions 1-61 or 98-111 of SEQ ID NO:4. In a further related aspect, the invention provides a nucleic acid fragment or oligonucleotide encoding a peptide comprised of at least 8 contiguous amino acids from positions 1-243 of SEQ ID NO:6. Particular embodiments of this aspect also include fragments or oligonucleotides comprised of at least 10, 15 or 20 amino acids. Also included within this aspect are any one of these fragments or oligonucleotides wherein the peptide comprises contiguous amino acids positions 1-61 or 98-111, or 161-170 of SEQ ID NO:6.

The invention also includes methods of use of the aforementioned nucleic acids. In one aspect, the invention provides a method of identifying a nucleic acid encoding all or a part of a metalloproteinase, comprising the steps of:(l) hybridizing a nucleic acid sample to the nucleic acids mentioned above and (2) identifying a sequence that hybridizes thereto. In a typical practice of this method, the step of identifying includes performing a polymerase chain reaction to amplify a sequence containing the sequence that hybridizes. Thus, the invention also includes a pair of primers that specifically amplifies all or a portion of a MMP-25 nucleic acid molecule.

In another aspect, the invention provides vectors containing MMP-25 and related sequences. More specifically, the invention provides a recombinant nucleic acid vector containing the aforementioned MMP-25 nucleic acid sequences. In a typical embodiment, the recombinant nucleic acid vector is an expression vector containing a promoter operably linked to the MMP-25 nucleic acid sequences. In another typical embodiment, the vector is selected from the group consisting of:plasmid vectors, phage vectors, herpes simplex viral vectors, adenoviral vectors, adenovirus-associated viral vectors and retroviral vectors. In a related aspect, the invention provides for a host cell containing any of the aforementioned vectors.

The vectors provided by the present invention are useful for producing MMP-25 polypeptides. Another aspect of the invention therefore includes a method of producing a MMP-25 polypeptide comprising the step of culturing a host cell comprising one of the aforementioned vectors, comprising a promoter operably linked to the MMP-25 sequence, under conditions and for a time sufficient to produce the MMP-25 polypeptide. In a preferred practice, the method further includes the step of purifying the MMP-25 polypeptide.

Accordingly, the invention also provides for a polypeptide comprising an amino acid sequence selected from the group consisting of: (a) the amino acid sequence according to SEQ ID NO:2, SEQ ID NO:4 or SEQ ID NO:6; (b) an amino acid sequence having at least 50% identity to the polypeptide of (a) or (b); (c) a sequence encoding a functional fragment of the polypeptide of (a) or (b); and (d) an amino acid sequence encoded by a nucleic acid that hybridizes under conditions of normal stringency to the foregoing. More typical embodiments of these polypeptides include those having at least 50%, 60%, 70%, 80%, 90%, or 95% identity to the polypeptide according to SEQ ID NO:2, SEQ ID NO:4 or SEQ ID NO:6. In particular embodiments, identity is calculated according a MEGALIGN algorithm using a gap penalty and gap length penalty each set at a value of 10.

The polypeptides of the present invention are useful for raising antibodies thereto which are specific for MMP-25 proteins. Accordingly, another aspect of the invention is an antibody that binds to a MMP, wherein said antibody specifically binds to one of the aforementioned polypeptides. In one embodiment, the antibody is a monoclonal antibody. Typically the antibody will bind to a type 25 MMP with a higher affinity than it binds to a non type 25 MMP. The antibody is also typically, a murine or human antibody. Related aspects include an antibody selected from the group consisting of F(ab′)₂, F(ab)₂, Fab Fab and Fv, and a hybridoma which produces the aforementioned monoclonal antibody.

Antibodies to MMP-25 polypeptides are useful in another aspect of the invention, which is a method of identifying a type 25 MMP polypeptide comprising incubating an antibody that binds to MMP-25 polypeptide with a sample containing protein for a time sufficient to permit said antibody to bind the type 25 MMP present in the sample. In a typical practice of this method, the antibody is bound to a solid support and optionally may be labeled.

In another aspect, the invention provides for fusion proteins containing a portion of a MMP-25 polypeptide which is useful for example, in raising antibodies to particular segments of a MMP-25 polypeptide. Accordingly the invention also includes a fusion protein, comprising a first MMP-25 polypeptide segment comprised of at least eight contiguous amino acids of a MMP-25 polypeptide, fused in-frame to a second polypeptide segment comprised of a non MMP-25 polypeptide. The size of the first polypeptide segment of the fusion protein is typically at least 10, 15, or 20 amino acids in length.

In a different aspect, the nucleic acid sequences of the present invention provide for derivative nucleic acids useful for modulating or inhibiting the expression of an MMP-25 polypeptide in a cell. More specifically, the invention provides for a ribozyme that cleaves RNA encoding the aforementioned MMP-25 polypeptides. This aspect also includes a nucleic acid molecule comprising a sequence that encodes such a ribozyme and a vector comprising said nucleic acid molecule. In a related aspect, the invention provides an antisense nucleic acid molecule comprising a sequence that is antisense to a portion of the MMP-25 nucleic acids described above, a vector comprising the antisense molecule, and vectors wherein the aforementioned ribozyme or antisense nucleic acid is operably linked to a promoter. Typical embodiments of these vectors are selected from the group consisting of plasmid vectors, phage vectors, herpes simplex viral vectors, adenoviral vectors, adenovirus-associated viral vectors and retroviral vectors. The invention also provides for a host cell comprising such a vector.

In yet another aspect, the invention provides a nucleic acid molecule comprising a sequence that encodes at a peptide of at least 27 amino acids in length, wherein said peptide is a consensus sequence for a Zn-binding domain of a MMP. Particular embodiments of this aspect includes SEQ ID NO:7 or SEQ ID NO:8 which are also useful for obtaining additional MMP sequences. Accordingly, the invention further provides a method of identifying a nucleic acid encoding all or a part of a MMP comprising, identifying a sequence encoded by the aforementioned consensus sequence, and cloning a sequence containing the identified sequence from a cDNA library.

In still another aspect, the MMP-25 sequences of the present invention provide for a method of inhibiting a catalytic activity of a MMP polypeptide in a cell comprising, administering an agent to the cell that inhibits a catalytic activity of the MMP, with the proviso that said agent inhibits the catalytic activity of a MMP-25 polypeptide to a greater extent than it inhibits the activity of at least one non-type 25 MMP. In a typical practice of this method, the MMP-25 polypeptide is preferentially expressed in the cell relative to the non-type 25 MMP. In one embodiment, the agent is topically administered to a skin cell of an animal. In a further embodiment of this aspect, the invention provides a method of inhibiting the expression of a metalloproteinase in a cell comprising administering to the cell, a vector comprising a nucleic acid means for inhibiting expression of a MMP-25 polypeptide. Embodiments of this method include nucleic means for expressing a non-functional variant of a MMP polypeptide selected from the group consisting of: (a) the amino acid sequence according to SEQ ID NO:2, SEQ ID NO:4 or SEQ ID NO:6; (b) an amino acid sequence having at least 50% identity to the polypeptide of (a) or (b); (c) a polypeptide comprised of a first MMP Zn-binding domain with the proviso that the polypeptide lacks a second MMP Zn-binding domain; and (d) an amino acid sequence encoded by a nucleic acid that hybridizes under conditions of high stringency to (a)-(c). In other embodiments of this method, the nucleic acid means comprises a ribozyme that cleaves an RNA encoding the MMP-25 polypeptide or comprises a molecule that is antisense to a portion of an RNA encoding the MMP-25 polypeptide.

In yet another aspect, the invention provides a method of reducing hair growth in a mammal comprising, applying a dermatologically acceptable composition comprising an inhibitor of a MMP, with the proviso that that the applied composition reduces the catalytic activity of a type 25 MMP to a greater extent than it reduces the catalytic activity of at least one non-type 25 MMP. In a preferred practice of this method, the inhibitor is selected to reduce the catalytic activity of the type 25 MMP to a greater extent than it reduces the catalytic activity of at least one non-type 25 MMP. In another practice, the inhibitor is applied in an amount that reduces the catalytic activity of the type 25 MMP to a greater extent than it reduces the catalytic activity of at least one non-type 25 MMP. In particular embodiments, the non-type 25 MMP is selected from the group consisting of MMP-2 and a MMP-9.

Another aspect of the present invention relates to the provision of a MMP sequence that has only one Zn-binding domain rather than the two normally associated with a MMP. In this aspect, the invention provides a polypeptide comprising a MMP of at least 471 amino acid residues in length, wherein said polypeptide is comprised of a first MMP Zn-binding domain and with the proviso that the polypeptide lacks a second MMP Zn-binding domain. In certain embodiments, the polypeptide exhibits a catalytic activity of a MMP. In another embodiment, the polypeptide will be non-functional and lack a catalytic activity making it useful for down regulating functional variants when expressed in the same cell.

### BRIEF DESCRIPTION OF THE DRAWINGS

FIG. 1 shows a 833 nucleic acid sequence encoding a portion of a matrix metalloproteinase 25 (MMP-25).

FIG. 2 shows a 1833 bp nucleic acid sequence (SEQ ID NO:5) which contains an open reading frame of 1539 bp (nucleotide position 12 to 1550 of SEQ ID NO:5) that encodes a full length MMP-25(l). The predicted 513 amino acid sequence (SEQ ID NO:6) of this full-length polypeptide is also included. The putative signal peptide and polyadenylation sequences are indicated by single underlining, a putative cysteine switch domain is indicated by boxed text and putative Zn-binding domains are indicated by double underlining.

FIG. 3 shows an amino acid sequence alignment between the two MMP-25 sequences, MMP-25(l) and MMP-25(s) (SEQ ID NO:5 and 3, respectively), in comparison to amino acid sequences of eighteen known MMPs (SEQ ID NOs:19-36). Positions for the leader peptide, cysteine switch and Zn-binding domains are indicated. Gaps introduced are indicated by “-” and residues that are identical to MMP-25(l) are indicated by “*”.

FIG. 4 shows a RT PCR analysis that illustrates a tissue expression pattern for MMP-25 in a panel of 36 different tissue samples.

FIG. 5 shows light micrographs from in-situ hybridization analysis that illustrate expression of MMP-25 in skin tissue, particularly follicle cells, more particularly in root sheath cells, and most particularly in the Henle layer. A-G:Antisense RNA probe for human MMP-25. H and I: Sense RNA probe for human MMP-25. Arrows in A, B, C, and D highlight cells in the hair follicle that express MMP-25 message. Cell nuclei are counterstained with H33258 in E, F, and G.

### DETAILED DESCRIPTION OF THE INVENTION

The following provides definitions of certain terms, and lists certain abbreviations used herein.

“Molecule” should be understood to include proteins or peptides (e.g., antibodies, recombinant binding partners, peptides with a desired binding affinity) nucleic acids (e.g., DNA, RNA, chimeric nucleic acid molecules, and nucleic acid analogues such as PNA); and organic or inorganic compounds.

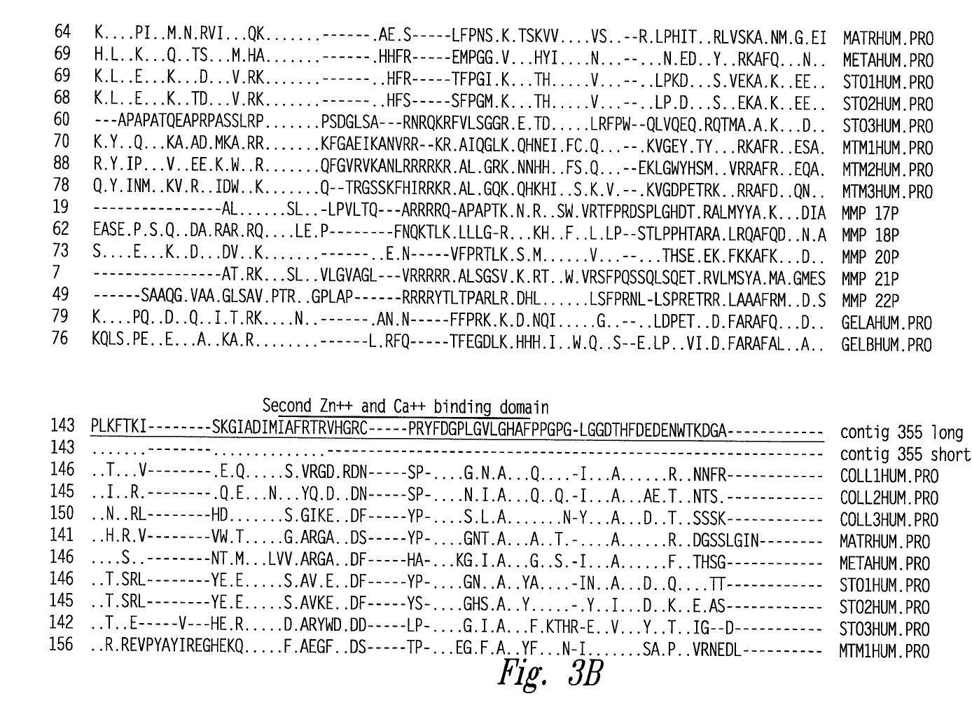

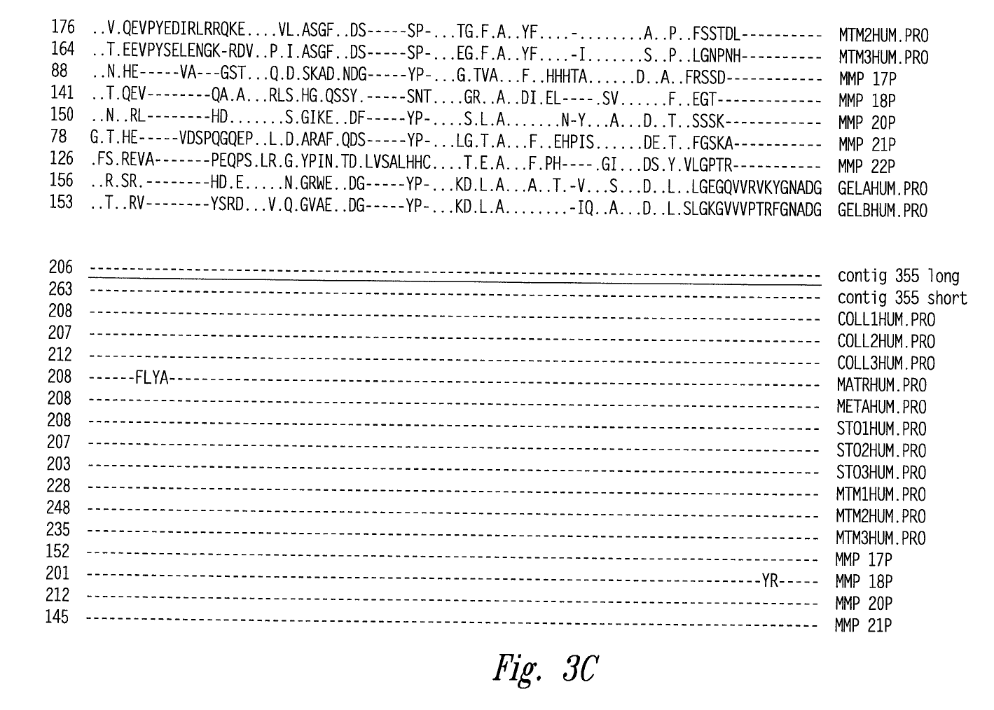

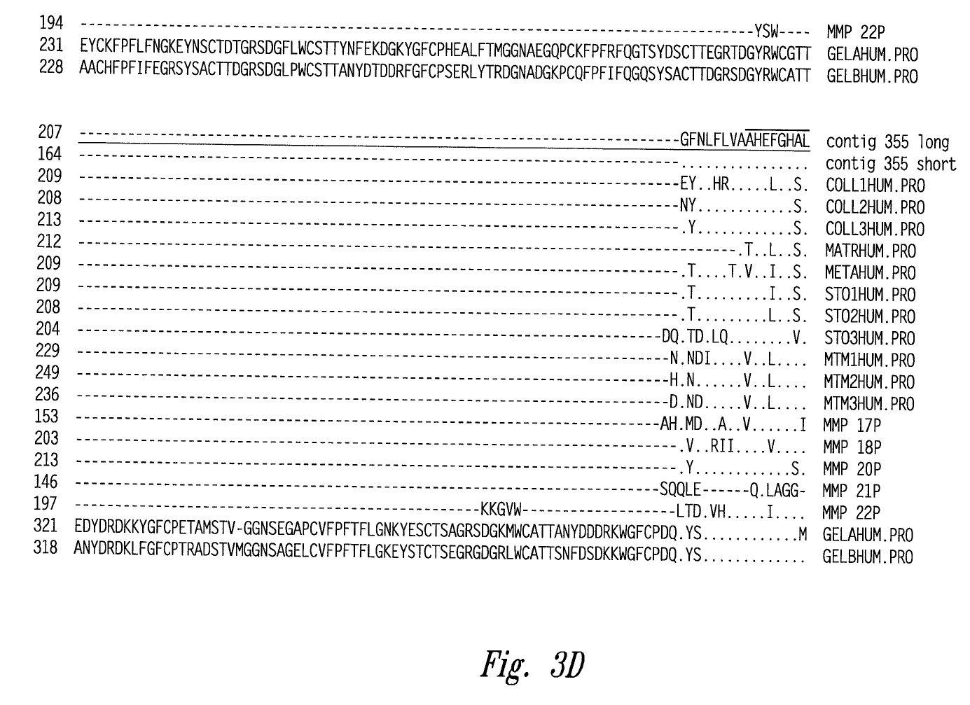

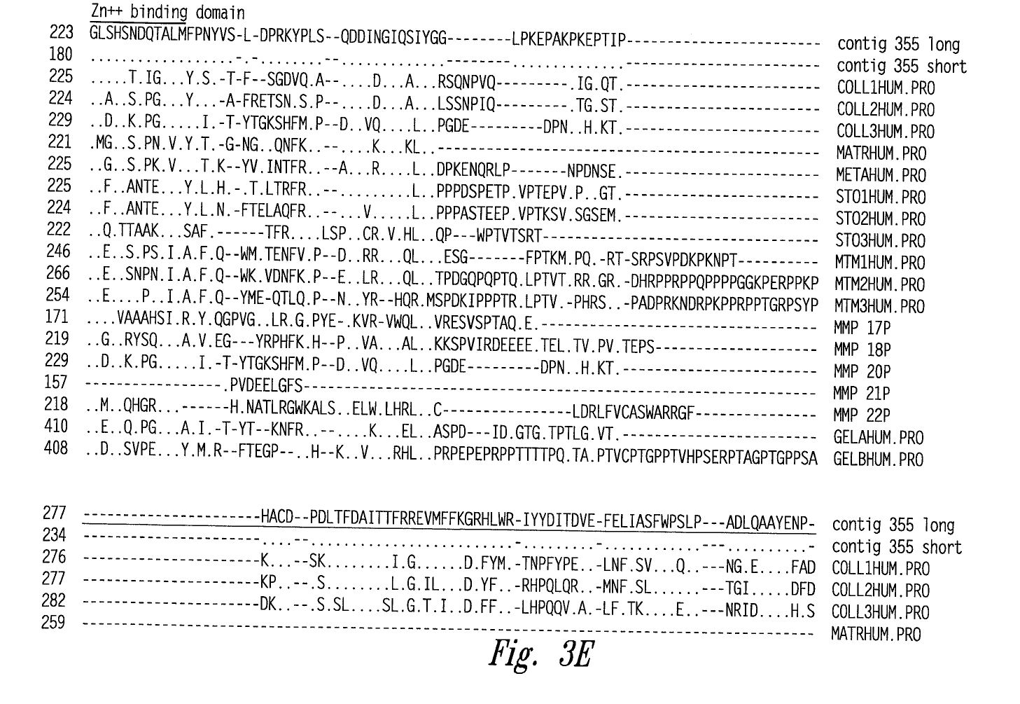

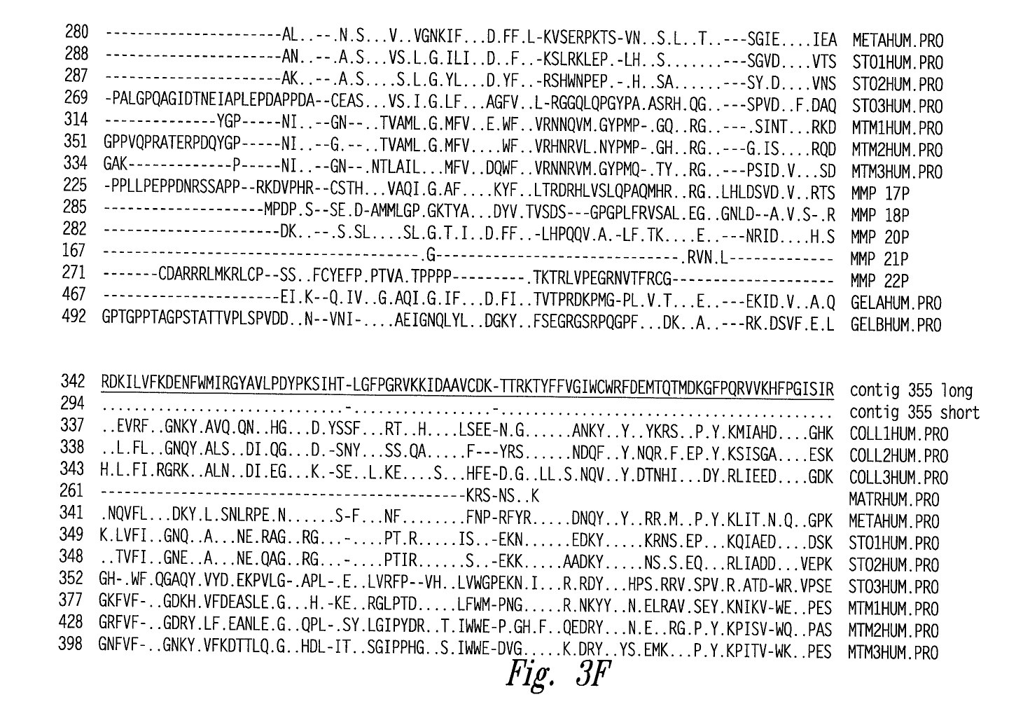

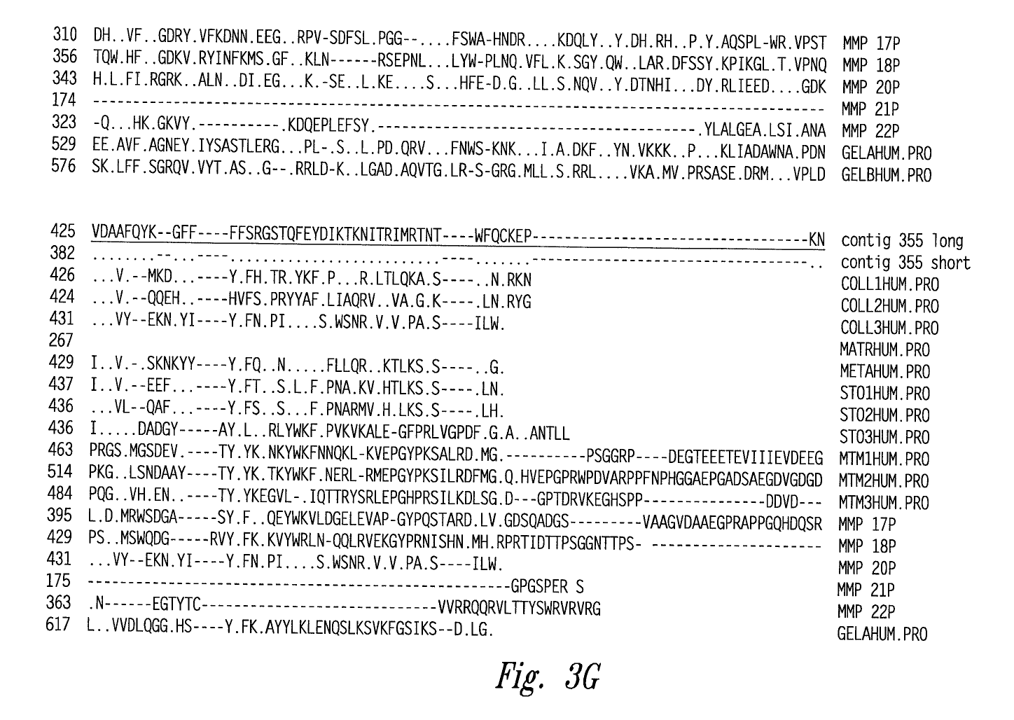

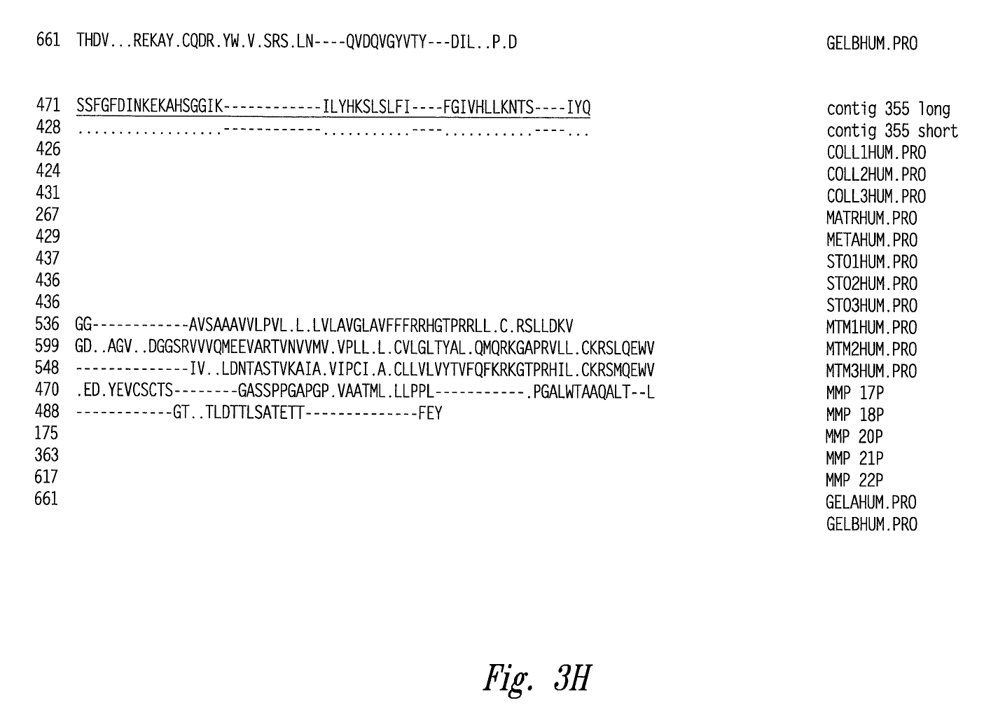

“MMP-25” or “Type 25 MMP” should be understood to include any polypeptide, or nucleic acid encoding a polypeptide of the MMP family, having at least 50%, 60%, 70%, 80%, 90%, or 95% amino acid identity to any one the polypeptides provided herein as SEQ ID NO:2, 4, or 6. These polypeptides will also have less than 50% sequence identity to known MMP members designated as MMP 1-3, or 7 -22. Example sequence comparisons and identity calculations are shown in Table 1 and FIG. 3.

“Non-type 25 MMP” refers to a polypeptide having less sequence identity to any of the MMPs according to SEQ ID NO:2, 4 or 6 than to another type of MMP, for example, MMPs 1-3 or 7-22. A non-type 25 MMP typically has less than 50% identity to any of the SEQ ID NO:2, 4 or 6.

“Vector” refers to an assembly that is capable of delivering a recombinant nucleic acid molecule to a cell wherein the nucleic acid molecule is maintained, either as part of an independently replicating element or as integrated into the genome of the cell. An “expression vector” is a vector that further includes transcriptional promoter elements operably linked to a recombinant nucleic acid of interest. The vector may be composed of either deoxyribonucleic acids (“DNA”), ribonucleic acids (“RNA”), or a combination of the two (e.g., a DNA-RNA chimeric). Optionally, the vector may include a polyadenylation sequence, one or more restriction sites, as well as one or more selectable markers such as neomycin phosphotransferase or hygromycin phosphotransferase. Additionally, depending on the host cell chosen and the vector employed, other genetic elements such as an origin of replication, additional nucleic acid restriction sites, enhancers, sequences conferring inducibility of transcription, and selectable markers, may also be incorporated into the vectors described herein.

An “isolated nucleic acid molecule” is a nucleic acid molecule that is not integrated in the genomic DNA of an organism. For example, a DNA molecule that encodes a MMP-25 polypeptide that has been separated from the genomic DNA of a eukaryotic cell is an isolated DNA molecule. Another example of an isolated nucleic acid molecule is a chemically-synthesized nucleic acid molecule that is not integrated in the genome of an organism. The isolated nucleic acid molecule may be genomic DNA, cDNA, RNA, or composed at least in part of nucleic acid analogs.

An “isolated polypeptide” is a polypeptide that has been removed by at least one step from its original environment. For example, a naturally occurring protein is isolated if it is separated from some or all of the coexisting material in the natural system such as carbohydrate, lipid, or other proteinaceous impurities associated with the polypeptide in nature. Within certain embodiments, a particular protein preparation contains an isolated polypeptide if it appears nominally as a single band on SDS-PAGE gel with Coomassie Blue staining.

A “functional fragment” of a MMP-25 polypeptide refers to a portion of a MMP-25 polypeptide that either (1) possesses a catalytic activity of a MMP-25 polypeptide, or (2) specifically binds with an anti-MMP-25 antibody.

“Humanized antibodies” are recombinant proteins in which murine complementarity determining regions of monoclonal antibodies have been transferred from heavy and light variable chains of the murine immunoglobulin into a human variable domain.

As used herein, an “antibody fragment” is a portion of an antibody such as F(ab′)₂, F(ab)₂, Fab′, Fab, and the like. Regardless of structure, an antibody fragment binds with the same antigen that is recognized by the intact antibody. For example, an anti-MMP-25 monoclonal antibody fragment binds with an epitope of MMP-25.

The term “antibody fragment” also includes any synthetic or genetically engineered protein that acts like an antibody by binding to a specific antigen to form a complex. For example, antibody fragments include isolated fragments consisting of the light chain variable region, “Fv” fragments consisting of the variable regions of the heavy and light chains, recombinant single chain polypeptide molecules in which light and heavy variable regions are connected by a peptide linker (“sFv proteins”), and minimal recognition units consisting of the amino acid residues that mimic the hypervariable region.

A “detectable label” is a molecule or atom which can be conjugated to an antibody moiety to produce a molecule useful for diagnosis. Examples of detectable labels include chelators, photoactive agents, radioisotopes, fluorescent agents, paramagnetic ions, enzymes, and other marker moieties.

An “immunoconjugate” is a molecule comprising an anti-MMP-25 antibody, or an antibody fragment, and a detectable label. An immunoconjugate has roughly the same, or only slightly reduced, ability to bind MMP-25 after conjugation as before conjugation.

“Preferentially expressed” means that an RNA or polypeptide encoded by the subject MMP sequence is detectable in one cell or tissue or cell type in a greater amount than it is detectable in a different cell or tissue type. For example, the MMP-25 sequences of the present invention are preferentially expressed in follicle cells and breast tissue over other cell types according to this meaning.

“Catalytic activity” of a matrix metalloproteinase is a measure of the ability of the matrix metalloproteinase to degrade one or more protein substrates. The catalytic activity of a subject matrix metalloproteinase may differ for different substrates.

“Expression of” a metalloproteinase means the synthesis of the subject metalloproteinase polypeptide in a cell by the processes of transcription into mRNA and/or translation of the mRNA into a protein, as those processes are ordinarily understood in the art. Similarly, a “pattern” of expression refers to the relative amounts of expression of a subject metalloproteinase in different cell types.

“Preferentially inhibited” means that the expression or catalytic activity of the subject type of MMP is reduced in a greater amount than the reduction of expression or catalytic activity of a different MMP exposed to the same conditions of inhibition.

“Moderate or stringent hybridization conditions” are conditions of hybridization of a probe nucleotide sequence to a target nucleotide sequence wherein hybridization will only be readily detectable when a portion of the target sequence is substantially similar to the complement of the probe sequence. Hybridization conditions vary with probe size as well as with temperature, time and salt concentration in a manner known to those of ordinary skill in the art. For example, moderate hybridization conditions for a 50 nucleotide probe would include hybridization overnight a buffer containing 5xSSPE (1XSSPE=180 mM sodium chloride, 10 mM sodium phosphate, 1 mM EDTA (pH 7.7), 5xDenhardt's solution (100xDenhardt's=2% (w/v) bovine serum albumin, 2% (w/v) Ficoll, 2% (w/v) polyvinylpyrrolidone) and 0.5% SDS incubated overnight at 55-60° C. Post-hybridization washes at moderate stringency are typically performed in 0.5xSSC (1xSSC=150 mM sodium chloride, 15 mM trisodium citrate) or in 0.5xSSPE at 55-60° C. Stringent hybridization conditions typically would include 2x SSPE overnight at 42° C., in the presence of 50% formamide followed by one or more washes in 0.1-0.2x SSC and 0.1% SDS at 65° C. for 30 minutes or more.

“Zn-binding domain” is a first peptide sequence within a MMP polypeptide that contains amino acid residues that enable the polypeptide to bind a zinc atom which binding is required to confer catalytic activity to the metalloproteinase. Typically, a Zn-binding domain contains a peptide of about 10-20 amino acids having the consensus sequence HExFHxxGxxHS/T (SEQ ID NO:17). Nineteen examples of Zn-binding domains are indicated in the sequences compared in FIG. 3.

“Zinc/calcium (Zn/Ca) binding domain” is a second peptide sequence within a MMP polypeptide that contains amino acid residues that enable the polypeptide to bind a Zn atom and which may also bind a calcium atom. Typically, a Zn-binding domain contains a peptide of about 10-20 amino acids having the consensus sequence HGxxxPxFDGxxG/AHAF (SEQ ID NO:37). Nineteen examples of Zn/Ca-binding domains are indicated in the sequences (SEQ ID NOs:5 and 19-36) compared in FIG. 3.

“Percent identity” or “% identity” with reference to a subject polypeptide or peptide sequence is the percentage value returned by comparing the whole of the subject polypeptide sequence to a test sequence using a computer implemented algorithm, typically with default parameters. Any program may be used, for example, BLAST, tBLAST or MEGALIGN. In a particular context, an algorithm is used with defined parameter settings such as with gap penalty and gap length penalty each set at a value of 10. An example of percent identity values determined using MEGALIGN with these particular parameters is shown in Table 1.

Abbreviations: MMP - matrix metalloproteinase; PCR—polymerase chain reaction; RT-PCR—PCR process in which RNA is first transcribed into DNA at the first step using reverse transcriptase (RT); cDNA—any DNA made by copying an RNA sequence into DNA form; EST—expressed sequence tag, which refers to an identified nucleotide sequence or fragment believed to be a part of an RNA that is expressed in a cell.

### NUCLEIC ACIDS.

Sequences

As mentioned above, the present invention provides for MMP sequences that encode a novel family of MMPs herein designated as MMP-25. Three representative nucleic acid sequences provided as SEQ ID NOs:1, 3; and 5 are molecules that encode MMP-25 polypeptides. The corresponding polypeptides are provided as SEQ ID NOs:2, 4 and 6 respectively.

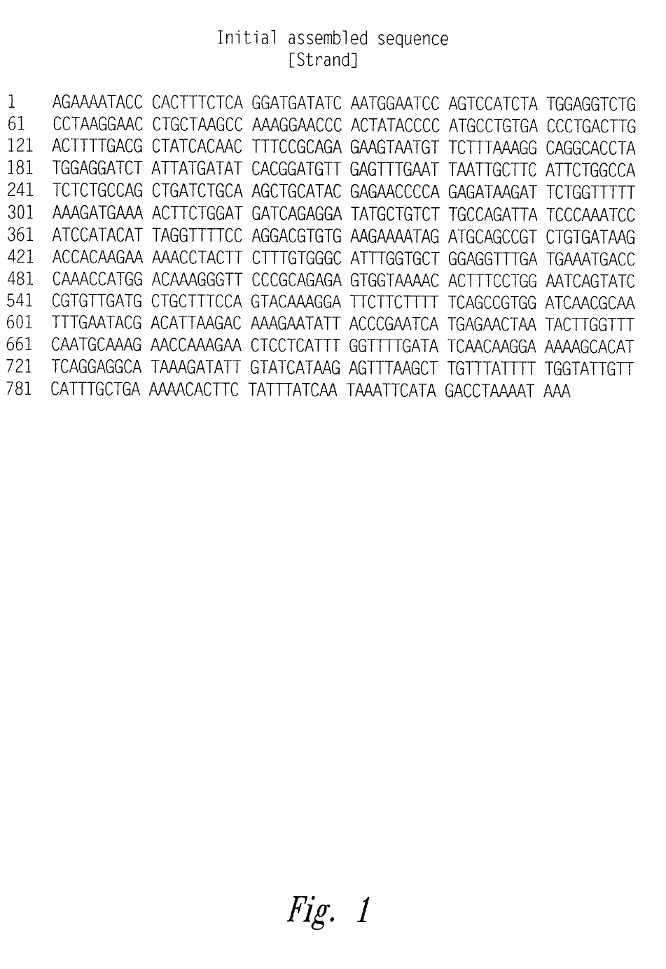

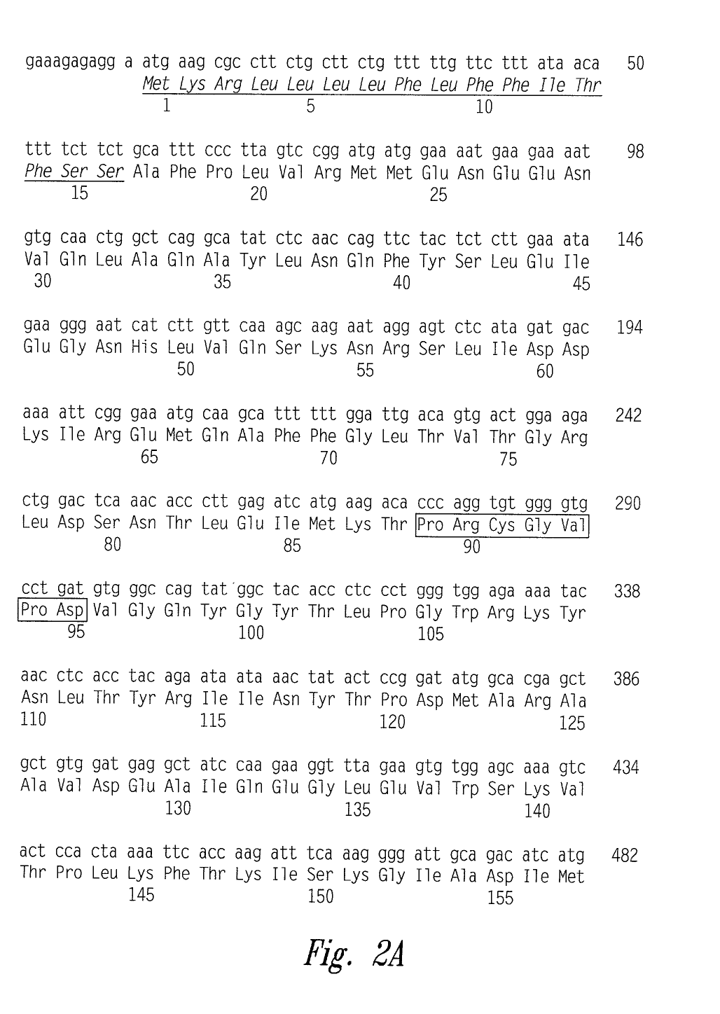

SEQ ID NO:1 is a 833 nucleotide fragment shown in FIG. 1 that encodes a portion (SEQ ID NO:2) of the MMP-25(l) polypeptide (SEQ ID NO:6). This polypeptide comprises a sequence having at least about 50% identity to two novel consensus sequences provided herein as SEQ ID NO:7 and 8. Each consensus sequence represents at least a 27 amino acid peptide domain determined to be representative of Zn-binding domains that occur in MMP polypeptides by aligning protein sequences of several MMP family members using a multiple sequence alignment program. It will be appreciated that polypeptides containing variations of these conserved peptides are not excluded from being potential MMPs on that basis alone. More particularly, nucleic acids encoding a polypeptide having at least 50% sequence identity to any one of the consensus sequences.

To obtain a full-length cDNA sequence for a novel MMP-25, a mammary gland cDNA library was screened by RT-PCR amplification using RACE reactions and a pair of primers comprised of contiguous nucleotides derived from SEQ ID NO:1 as described in more detail in Example 1. A cDNA of 1833 nucleotides that includes a 1539 open reading frame was obtained (SEQ ID NO:5). The 833 nucleotide sequence according to SEQ ID NO:1 is entirely contained within SEQ ID NO:5 and corresponds to positions 741-1573 thereof. Translation of the open reading frame of SEQ ID NO:5 provided a polypeptide of about 54 kD comprising 513 amino acids provided here as SEQ ID NO:6. The polypeptide fragment according to SEQ ID NO:2 (which is encoded by SEQ ID NO:1) corresponds to amino acid positions 244-513 of SEQ ID NO:6. Therefore, positions 1-243 of SEQ ID NO:6 are not found in the polypeptide encoded by SEQ ID NO:1.

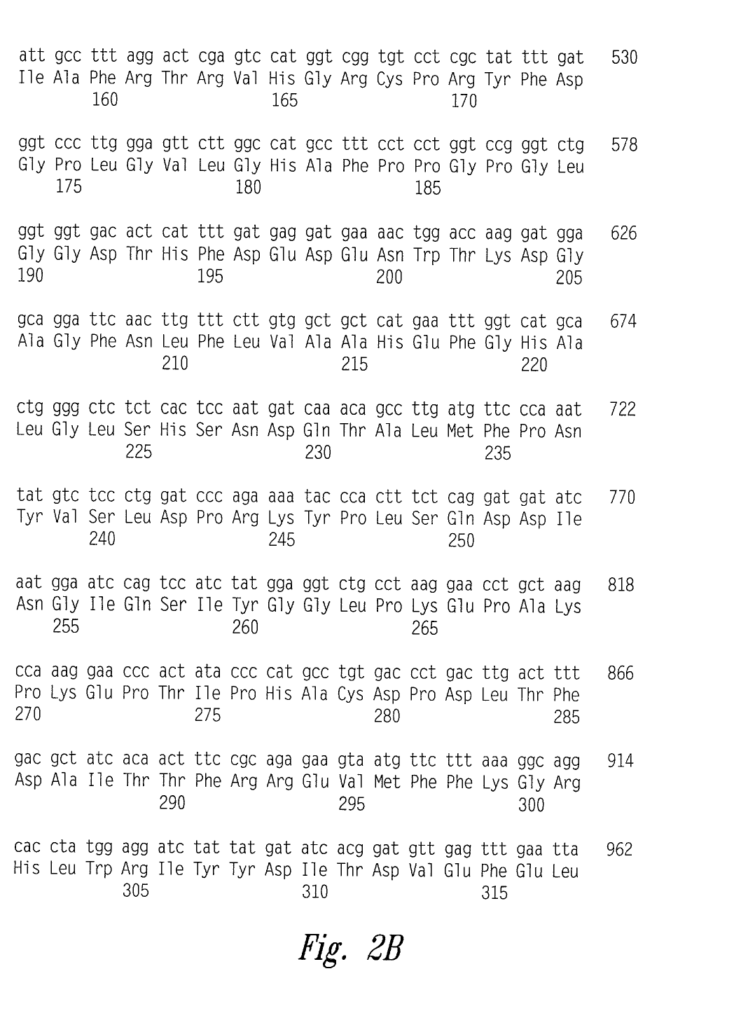

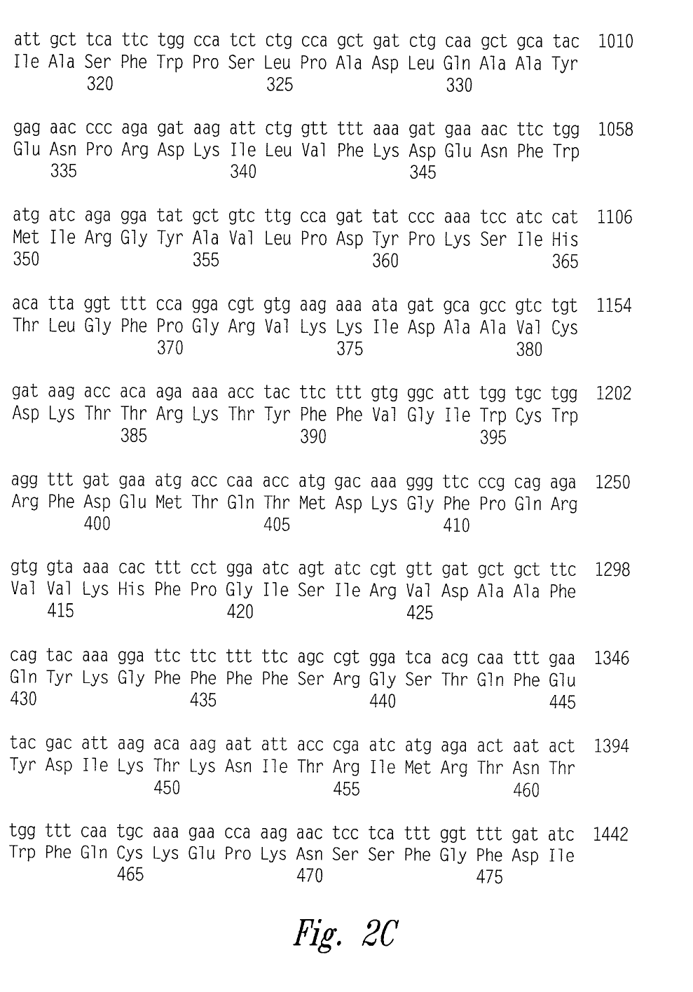

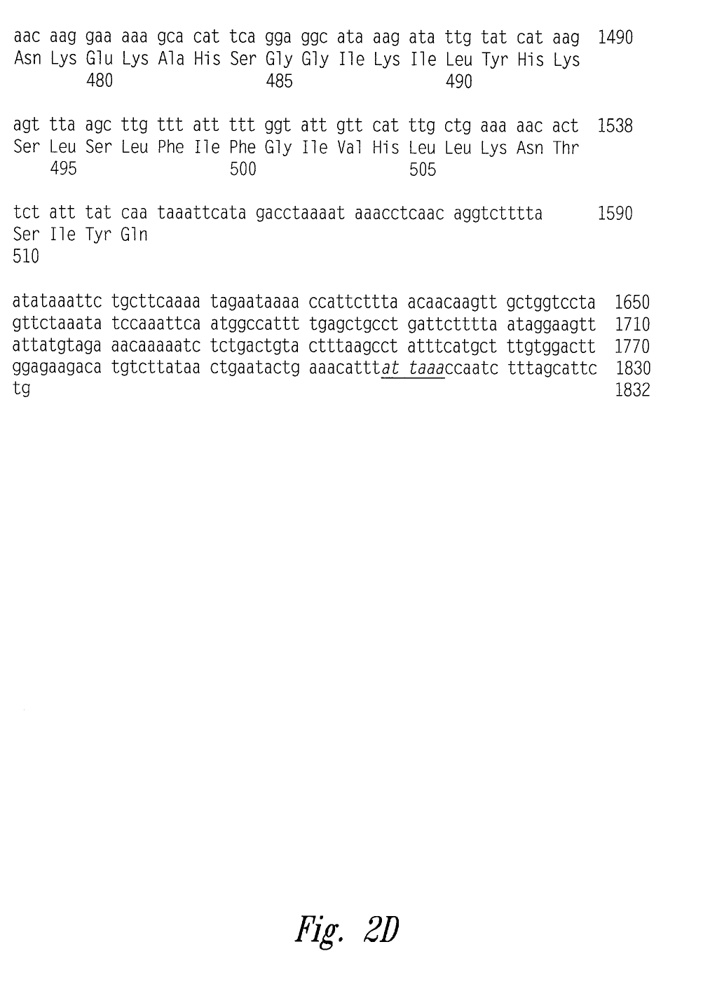

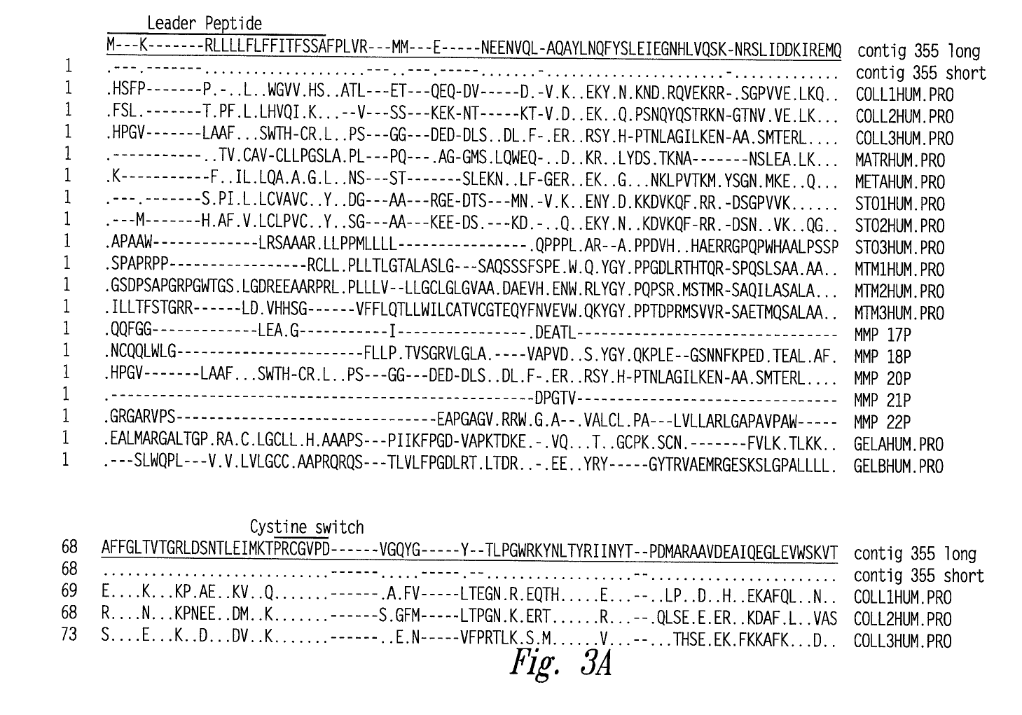

FIG. 2 shows the obtained 1833 nucleotides (SEQ ID NO:5), along with the translated open reading frame according to SEQ ID NO:6, and illustrates other features of these sequences. The polypeptide, herein designated MMP-25(l), contains several domains characteristic of the MMP gene family. These include a signal peptide, a pro-peptide, a first Zn-binding domain, a second Zn/Ca-binding domain, a hemopexin domain, and a cysteine-switch sequence (PCGVPD, SEQ ID NO:18) located within the pro-peptide.

In addition, to MMP-25(l), a second MMP-25 family member herein designated as MMP-25(s) was isolated by screening a library by RT-PCR as described in Example 2. The nucleic acid sequence of MMP-25(s) is provided as SEQ ID NO:3 and the translated open reading frame encoding a 470 amino acid polypeptide is provided as SEQ ID NO:4. The polypeptide of MMP-25(s) is identical to MMP-25(l) except that it is missing 43 amino acid residues in a region of the protein that corresponds to the Zn/Ca-binding domain. The conserved regions of both Zn-binding domains varied from the consensus sequences first used for the search. Therefore, use of Zn-binding domain consensus sequences are useful for identifying divergent MMPs so long as the MMP sequence contains at least one sequence having at least about 50% identity with the consensus sequences.

Despite conservation in the aforementioned polypeptide domain regions, the remainder of the MMP-25 sequences show low similarity to other MMP family members. Sequence identity was calculated as a percentage using the MEGALIGN algorithm provided with sequence alignment program DNASTAR (Madison, Wiss.) using a Clustal method with the gap penalty and gap length penalty each set at 10. Gaps were established to maximize the number of sequence matches between the MMP-25(l) source (SEQ ID NO:5) and other MMP query sequences (SEQ ID NOs 19-36). The results are shown in Table 1.

TABLE 1
Percent Amino Acid Sequence Identity of MMP-25(1)
to Other MMP Sequences

| | | Percent Identity |
|---|---|---|
| MMP | Names indicated in FIG. 3 | to MMP-25(1) |
| MMP-25(s)* | Contig 355 short form | 99.2 |
| MMP-1 | COLL1.HUM.PRO | 45.0 |
| MMP-8 | COLL2.HUM.PRO | 44.5 |
| MMP-13 | COLL3.HUM.PRO | 43.5 |
| MMP-7 | MATRHUM.PRO | 39.7 |
| MMP-12 | METAHUM.PRO | 43.2 |
| MMP-3 | STO1HUM.PRO | 46.8 |
| MMP-10 | STO2HUM.PRO | 46.6 |
| MMP-11 | STO3HUM.PRO | 24.2 |
| MMP-14 | MTM1HUM.PRO | 26.3 |
| MMP-15 | MTM2HUM.PRO | 27.1 |
| MMP-16 | MTM3HUM.PRO | 26.1 |
| MMP-17P | 17P | 22.0 |
| MMP-18P | 18P | 22.6 |
| MMP-20P | 20P | 43.5 |
| MMP-21P | 21P | 18.6 |
| MMP-22P | 22P | 16.9 |
| MMP-2 | GELAHUM.PRO | 31.6 |
| MMP-9 | GELBHUM.PRO | 23.2 |

*MMP-25(s) is provided herein

The highest overall sequence identity to any other known MMP is 46% to members of the stomelysin subfamily of MMPs which include MMP3, MMP10 and MMP11. A comparison of MMP-25(1) to other sequences using a different sequence comparison algorithm, namely Blastn or Blastp, also shows MMP-25 sequences to have low sequence identity with respect to other known MMP. More specifically, the greatest sequence identity obtained was 58% to a Gallus gallus MMP sequence. These programs were run using default settings. However these programs do not return an identity score that evaluates the whole of the MMP-25 sequence, but only evaluates those portions of MMP-25 sequences where some level of identity to the comparison sequence can be found. Typically, there is no significant identity to other MMP sequences in the region corresponding to positions 481-510 of SEQ ID NO:6 (which corresponds to positions 438-470 of SEQ ID NO:4). Accordingly, the overall sequence identity of MMP-25 to other known sequences is less than 50% when the whole of the MMP-25 sequence is compared to a other MMP sequences using BLAST programs as well as MEGALIGN.

FIG. 3 illustrates patterns of sequence identity between the MMP-25 sequences of the present invention in comparison to eighteen other known MMP sequences. The comparison indicates regions where sequence identity is high, which include the aforementioned domains common amongst MMP proteins which are also depicted FIG. 3. In addition, FIG. 3 indicates that there are regions of low identity between MMP-25 and other MMP sequences. Regions of low identity are particularly useful for identifying MMP-25 family members by hybridization or antibody techniques as described in more detail herein. Regions of low identity to MMP-25(l) include positions 1-61, 98-111, 161-170, and 261-570 of SEQ ID NO:6 and regions of low identity to MMP-25(s) include positions 1-61, 98-111, and 218-470 of SEQ ID NO:4. As noted above, SEQ ID NO:4 is missing 43 amino acids within the second Zn/Ca-binding domain. It is surprising to further note that position 161-170 of SEQ ID NO:6 has low similarity to other MMP sequences although this segment is part of the Zn/Ca-binding domain such as would be common among MMP proteins.

Variants

Sequences that are variants of the aforementioned sequences that encode other members of the MMP-25 family are also provided. More specifically, in addition to the isolated nucleic acids comprising nucleotide sequences according to SEQ ID NO:1 or SEQ ID NO:3; or SEQ ID NO:5; sequences that hybridize under conditions of normal to high stringency to the above sequences are also provided. Preferred sequences are those that hybridize under conditions of high stringency. Similarly, variant nucleic acid sequences of the MMP-25 family include those encoding a polypeptide according to SEQ ID NO:2, SEQ ID NO:4 or SEQ ID NO:6; those sequences encoding a polypeptide having at least 50% identity to these polypeptide and those encoding a functional fragment of these polypeptides. Preferred nucleic acid variants are those encoding a polypeptides having at least 60%, 70%, 80%, 90%, or 95% identity to the aforementioned amino acid sequences. Sequences that are the compliment or the above sequences are also included.

As used herein, two amino acid sequences have “100% identity” if the amino acid residues of the two amino acid sequences are the same when aligned for maximal correspondence. Similarly, two nucleotide sequences have “100% nucleotide sequence identity” if the nucleotide residues of the two nucleotide sequences are the same when aligned for maximal correspondence. Sequence comparisons can be performed using standard software programs such as BLAST or MEGALIGN mentioned above Still others include those provided in the LASERGENE bioinformatics computing suite, which is produced by DNASTAR (Madison, Wis.). Reference for algorithms such as ALIGN or BLAST may be found for example, in Altschul, J. Mol. BioL 219:555-565, 1991; Henikoff and Henikoff, Proc. Natl. Acad. Sci. USA 89:10915-10919, 1992) BLAST is available at the NCBI website (http://www/ncbi.nlm.nih.gov/cgi-bin/BLAST). Default parameters may be used. Other methods for comparing two nucleotide or amino acid sequences by determining optimal alignment are well-known to those of skill in the art (see, for example, Peruski and Peruski, The Internet and the New Biology:Tools for Genomic and Molecular Research (ASM Press, Inc. 1997), Wu et al. (eds.), “Information Superhighway and Computer Databases of Nucleic Acids and Proteins,” in Methods in Gene Biotechnology, pages 123-151 (CRC Press, Inc. 1997), and Bishop (ed.), Guide to Human Genome Computing, 2nd Edition (Academic Press, Inc. 1998)).

These variant sequences include members of the MMP-25 family that retain structural and functional characteristics more similar to the MMP-25 sequence of the present invention than to non-type 25 MMP family members such as MMP 1-3, or 7-22. These variants include naturally-occurring polymorphisms or allelic variants of MMP-25 genes, MMP-25 genes that are divergent across species, as well as synthetic genes that contain conservative amino acid substitutions of these amino acid sequences. Additional variant forms of a MMP-25 gene are nucleic acid molecules that contain insertions or deletions of the nucleotide sequences described herein.

As mentioned, a variant MMP-25 polypeptide should have at least 50% amino acid sequence identity to SEQ ID NO:2, 4 or 6. Regardless of the particular method used to identify a MMP-25 variant gene or variant MMP-25, a variant MMP-25 or a polypeptide encoded by a variant MMP-25 gene can be functionally characterized by, for example, its ability to bind specifically to an anti-MMP-25 antibody or its ability to degrade the same panel of substrates with the same relative catalytic activity as the aforementioned MMP-25 polypeptides.

Variants also include functional fragments of MMP-25 genes. Within the context of this invention, a “functional fragment” of a MMP-25 gene refers to a nucleic acid molecule that encodes a portion of a MMP-25 polypeptide which either (1) possesses the above-noted functional activity, or (2) specifically binds with an anti-MMP-25 antibody. For example, the MMP-25 polypeptide encoded by the 833 nucleotide fragment (SEQ ID NO:2) is a functional fragment of the larger MMP-25 disclosed above as SEQ ID NO 6.

Fragments and oligonucleotides

Also provided herein are nucleic acid fragments or oligonucleotides useful as probes and primers for identifying or obtaining MMP-25 sequences. More specifically, a nucleic acid fragment or oligonucleotide should comprise at least 15 contiguous nucleotides of SEQ ID NO:1 or SEQ ID NO:3, or SEQ ID NO:5 with the proviso that said nucleic acid fragment is not SEQ ID NO:15 or 16. More particular embodiments include fragments or oligonucleotides such as positions 1-653 of SEQ ID NO:3 or 1-741 or 1573-1841 of SEQ ID NO:5. Particular embodiments of these nucleic acid fragments or oligonucleotides include any of the above where the length is at least 18, 24, 30, 50 or greater than 50 nucleotides. Complements of the above sequences are also included.

Another embodiment of nucleic acid fragments or oligonucleotides of this invention include those that encode a peptide epitope that can be detected, for example, by the ability to specifically bind to a MMP-25 antibody or which can be used to elicit an immune response in an animal. Useful peptide epitopes are those capable of eliciting antibodies that specifically bind to the peptide or polypeptide comprised of the same, or that are capable of eliciting a T-cell response. Peptide sequences of 8 or more amino acids are useful in this regard since it is generally understood by those skilled in the art that 8 amino acids is the lower size limit for a peptide to interact with the major histocompatibility complex (MHC). More preferred embodiments include nucleic acid fragments or oligonucleotides encoding at least 10, 15 or 20 amino acids.

Therefore, the present invention provides for nucleic acid fragments or oligonucleotides encoding a peptide comprised of at least 8 contiguous amino acids of the sequence according to SEQ ID NO:2, SEQ ID NO:4 or SEQ ID NO:6, with the proviso that said nucleic acid fragment is not SEQ ID NO:15 or 16. Particular embodiments of this aspect include nucleic acid fragments or oligonucleotides encoding a peptide comprised of at least 10, 15, or 20 amino acids. Still more particular embodiments include nucleic acid fragments wherein the encoded peptide comprises sequences particularly distinctive of MMP-25 polypeptides. These include sequence such as those encoding peptides from positions 1-243 of SEQ ID NO:6. Other preferred sequences that are distinctive of MMP-25 include those encoding peptides from positions 1-61, 98-111, 161-170 or 261-513 of SEQ ID NO:6. Also included in this regard are nucleic acids encoding at least 8, 10, 15 or 20 amino acids from positions 1-200 of SEQ ID NO:4, with preferred fragments or oligonucleotides encoding a peptide from positions 1-61 or 98-111 of SEQ ID NO:4.

Methods of use of nucleic acids, fragments and oligonucleotides

The aforementioned nucleic acids fragments and oligonucleotides are useful for the identification or isolation of MMP-25 nucleic acids, polypeptides and variants thereof. Typically, the nucleic acid fragments are used for probes for hybridization to sample sequences or as primers for PCR reactions. Thus, the invention provides for methods of identifying a nucleic acid encoding all or a part of a metalloproteinase, comprising the steps of:(1) hybridizing a nucleic acid sample to the nucleic acids mentioned above and (2) identifying a sequence that hybridizes thereto. In a typical practice of this method, the step of identifying includes performing a polymerase chain reaction to amplify a sequence containing the sequence that hybridizes. Thus the invention also includes at least one pair of primers that specifically amplifies all or a portion of a MMP-25 nucleic acid molecule.

In addition, as discussed above, the present invention includes consensus sequences for a Zn or Zn/Ca-binding domain of MMPs. The consensus sequences used are unique, and permit identification and isolation of MMP sequences having at least 50% identity to the consensus sequences. Therefore, another aspect of the present invention provides a nucleic acid comprising a sequence that encodes a peptide of at least 27 amino acids in length, wherein said peptide is a consensus sequence for a Zn-binding domain of a MMP. Particular embodiments of this aspect include SEQ ID NO:7 or SEQ ID NO:8. In a related aspect, the invention provides a general method of identifying a nucleic acid encoding all or a part of a MMP that includes the steps of identifying a sequence encoded by the aforementioned consensus sequences, and cloning a sequence containing the identified sequence from a cDNA library.

### Identification and Isolation of MMP-25 nucleic acids

DNA molecules encoding a gene can be obtained by screening a human CDNA or genomic library using polynucleotide probes based upon the aforementioned MMP-25 sequences, fragments and oligonucleotides.

For example, the first step in the preparation of a cDNA library is to isolate RNA using methods well-known to those of skill in the art. In general, RNA isolation techniques provide a method for breaking cells, a means of inhibiting RNase-directed degradation of RNA, and a method of separating RNA from DNA, protein, and polysaccharide contaminants. For example, total RNA can be isolated by freezing tissue in liquid nitrogen, grinding the frozen tissue with a mortar and pestle to lyse the cells, extracting the ground tissue with a solution of phenol/chloroform to remove proteins, and separating RNA from the remaining impurities by selective precipitation with lithium chloride (see, for example, Ausubel et al. (eds.), Short Protocols in Molecular Biology, 3rd Edition, pages 4-1 to 4-6 (John Wiley & Sons 1995) [“Ausubel (1995)”]; Wu et al., Methods in Gene Biotechnology, pages 33-41 (CRC Press, Inc. 1997) [“Wu (1997)”]).

Alternatively, total RNA can be isolated by extracting ground tissue with guanidinium isothiocyanate, extracting with organic solvents, and separating RNA from contaminants using differential centrifugation (see, for example, Ausubel (1995) at pages 4-1 to 4-6; Wu (1997) at pages 33-41).

In order to construct a CDNA library, poly(A)⁺RNA is isolated from a total RNA preparation. Poly(A)⁺RNA can be isolated from total RNA by using the standard technique of oligo(dT)-cellulose chromatography (see, for example, Ausubel (1995) at pages 4-11 to 4-12).

Double-stranded CDNA molecules are synthesized from poly(A)⁺RNA using techniques well known to those in the art. (see, for example, Wu (1997) at pages 41-46). Commercially available kits can be used to synthesize double-stranded CDNA molecules. For example, such kits are available from Life Technologies, Inc. (Gaithersburg, Maryland), Clontech Laboratories, Inc. (Palo Alto, Calif.), Promega Corporation (Madison, Wis.) and Stratagene Cloning Systems (La Jolla, Calif.).

The basic approach for obtaining MMP-25 CDNA clones can be modified by constructing a subtracted cDNA library which is enriched in MMP CDNA molecules. Techniques for constructing subtracted libraries are well-known to those of skill in the art (see, for example, Sargent, “Isolation of Differentially Expressed Genes,” in Meth. Enzymol. 152:423, 1987, and Wu et al. (eds.), “Construction and Screening of Subtracted and Complete Expression cDNA Libraries,” in Methods in Gene Biotechnology, pages 29-65 (CRC Press, Inc. 1997)).

Various cloning vectors are appropriate for the construction of a CDNA library. For example, a CDNA library can be prepared in a vector derived from bacteriophage, such as a λgt10 vector (see, for example, Huynh et al., “Constructing and Screening cDNA Libraries in λgt10 and λgt11,” in DNA Cloning:A Practical Approach Vol. I, Glover (ed.), page 49 (IRL Press, 1985); Wu (1997) at pages 47-52).

Alternatively, double-stranded cDNA molecules can be inserted into a plasmid vector, such as a pBluescript vector (Stratagene Cloning Systems; La Jolla, Calif.), a LambdaGEM-4 (Promega Corp.; Madison, Wis.) or other commercially available vectors. Suitable cloning vectors also can be obtained from the American Type Culture Collection (Rockville, Md.).

In order to amplify the cloned cDNA molecules, the cDNA library is inserted into a prokaryotic host, using standard techniques. For example, a cDNA library can be introduced into competent E. coli DH5 cells, which can be obtained from Life Technologies, Inc. (Gaithersburg, Md.).

A human genomic DNA library can be prepared by means well-known in the art (see, for example, Ausubel (1995) at pages 5-1 to 5-6; Wu (1997) at pages 307-327). Genomic DNA can be isolated by lysing tissue with the detergent Sarkosyl, digesting the lysate with proteinase K, clearing insoluble debris from the lysate by centrifugation, precipitating nucleic acid from the lysate using isopropanol, and purifying resuspended DNA on a cesium chloride density gradient.

DNA fragments that are suitable for the production of a genomic library can be obtained by the random shearing of genomic DNA or by the partial digestion of genomic DNA with restriction endonucleases. Genomic DNA fragments can be inserted into a vector, such as a bacteriophage or cosmid vector, in accordance with conventional techniques, such as the use of restriction enzyme digestion to provide appropriate termini, the use of alkaline phosphatase treatment to avoid undesirable joining of DNA molecules, and ligation with appropriate ligases. Techniques for such manipulation are well-known in the art (see, for example, Ausubel (1995) at pages 5-1 to 5-6; Wu (1997) at pages 307-327).

Nucleic acid molecules that encode a MMP-25 gene can also be obtained using the polymerase chain reaction (PCR) with oligonucleotide primers having nucleotide sequences that are based upon the nucleotide sequences of the human MMP-25 gene, as described herein. General methods for screening libraries with PCR are provided by, for example, Yu et al., “Use of the Polymerase Chain Reaction to Screen Phage Libraries,” in Methods in Molecular Biology, Vol. 15:PCR Protocols:Current Methods and Applications, White (ed.), pages 211-215 (Humana Press, Inc. 1993). Techniques for using PCR to isolate related genes are described by, for example, Preston, “Use of Degenerate Oligonucleotide Primers and the Polymerase Chain Reaction to Clone Gene Family Members,” in Methods in Molecular Biology, Vol. 15:PCR Protocols:Current Methods and Applications, White (ed.), pages 317-337 (Humana Press, Inc. 1993). Examples 1 and 2 illustrate one approach to obtaining MMP-25 nucleic acids using RT-PCR.

Alternatively, human genomic libraries can be obtained from commercial sources such as Research Genetics (Huntsville, AL) and the American Type Culture Collection (Rockville, Md.).

A library containing cDNA or genomic clones can be screened with one or more polynucleotide probes based upon SEQ ID NO:1, 3,or 5 using standard methods (see, for example, Ausubel (1995) at pages 6-1 to 6-11).

Anti-MMP-25 antibodies, produced as described below, can also be used to isolate DNA sequences that encode MMP-25 genes from cDNA libraries. For example, the antibodies can be used to screen λgt11 expression libraries, or the antibodies can be used for immunoscreening following hybrid selection and translation (see, for example, Ausubel (1995) at pages 6-12 to 6-16; Margolis et al., “Screening β expression libraries with antibody and protein probes,” in DNA Cloning 2:Expression Systems, 2nd Edition, Glover et al. (eds.), pages 1-14 (Oxford University Press 1995)).

The sequence of a MMP-25 CDNA or MMP-25 genomic fragment can be determined using standard methods. The identification of genomic fragments containing a MMP-25 promoter or regulatory element can be achieved using well-established techniques, such as deletion analysis (Ausubel (1995)).

A MMP-25 gene can also be obtained by synthesizing DNA molecules using mutually priming long oligonucleotides and the nucleotide sequences described herein (Ausubel (1995) at pages 8-8 to 8-9). Established techniques using the polymerase chain reaction provide the ability to synthesize DNA molecules at least two kilobases in length (Adang et al., Plant Molec. Biol. 21:1131, 1993; Bambot et al., PCR Methods and Applications 2:266, 1993; Dillon et al., “Use of the Polymerase Chain Reaction for the Rapid Construction of Synthetic Genes,” in Methods in Molecular Biology, Vol. 15:PCR Protocols:Current Methods and Applications, White (ed.), pages 263-268 (Humana Press, Inc. 1993); Holowachuk et al., PCR Methods Appl. 4:299, 1995).

### Production of Variants

Nucleic acid molecules encoding variant MMP-25 nucleic acids can be produced by screening various cDNA or genomic libraries with polynucleotide probes having nucleotide sequences based upon SEQ ID NO:1, 3 or 5 and the fragments or oligonucleotides derived therefrom described above. MMP-25 nucleic acids and variants can also be constructed synthetically. For example, a nucleic acid molecule can be obtained that encodes a polypeptide having a conservative amino acid change, compared with the amino acid sequence of SEQ ID NO:2, 4, or 6, That is, variants can be obtained that contain one or more amino acid substitutions of SEQ ID NO:2, 4, or 6, in which an alkyl amino acid is substituted for an alkyl amino acid in a MMP-25 amino acid sequence, an aromatic amino acid is substituted for an aromatic amino acid in a MMP-25 amino acid sequence, a sulfur-containing amino acid is substituted for a sulfur-containing amino acid in a MMP-25 amino acid sequence, a hydroxy-containing amino acid is substituted for a hydroxy-containing amino acid in a MMP-25 amino acid sequence, an acidic amino acid is substituted for an acidic amino acid in a MMP-25 amino acid sequence, a basic amino acid is substituted for a basic amino acid in a MMP-25 amino acid sequence, or a dibasic monocarboxylic amino acid is substituted for a dibasic monocarboxylic amino acid in a MMP-25 amino acid sequence.

Among the common amino acids, for example, a “conservative amino acid substitution” is illustrated by a substitution among amino acids within each of the following groups:(l) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. In making such substitutions, it is important to, where possible, maintain the cysteine backbone outlined in FIG. 1.

Conservative amino acid changes in a MMP-25 proteins can be introduced by substituting nucleotides for the nucleotides recited in SEQ ID NO:1, 3 or 5. Such “conservative amino acid” variants can be obtained, for example, by oligonucleotide-directed mutagenesis, linker-scanning mutagenesis, mutagenesis using the polymerase chain reaction, and the like (see Ausubel (1995) at pages 8-10 to 8-22; and McPherson (ed.), Directed Mutagenesis:A Practical Approach (IRL Press 1991)). The functional ability of such variants can be determined using a standard method, such as the zymographic assay described in Example 5. Alternatively, a variant MMP-25 polypeptide can be identified by the ability to specifically bind anti-MMP-25 antibodies.

Routine deletion analyses of nucleic acid molecules can be performed to obtain “functional fragments” of a nucleic acid molecule that encodes a MMP-25 polypeptide. As an illustration, DNA molecules having the nucleotide sequence of SEQ ID NO:1, 3 or 5 can be digested with Bal31 nuclease to obtain a series of nested deletions. The fragments are then inserted into expression vectors in proper reading frame, and the expressed polypeptides are isolated and tested for activity, or for the ability to bind anti-MMP-25 antibodies. One alternative to exonuclease digestion is to use oligonucleotide-directed mutagenesis to introduce deletions or stop codons to specify production of a desired fragment. Alternatively, particular fragments of a MMP-25 gene can be synthesized using the polymerase chain reaction.

Standard techniques for functional analysis of proteins are described by, for example, Treuter et al., Molec. Gen. Genet. 240:113, 1993; Content et al., “Expression and preliminary deletion analysis of the 42 kD 2-5A synthetase induced by human interferon,” in Biological Interferon Systems, Proceedings of ISIR-TNO Meeting on Interferon Systems, Cantell (ed.), pages 65-72 (Nijhoff 1987); Herschman, “The EGF Receptor,” in Control of Animal Cell Proliferation, Vol. 1, Boynton et al., (eds.) pages 169-199 (Academic Press 1985); Coumailleau et al., J. Biol. Chem. 270:29270, 1995; Fukunaga et al., J. Biol. Chem. 270:25291, 1995; Yamaguchi et al., Biochem. Pharmacol. 50:1295, 1995; and Meisel et al., Plant Molec. Biol. 30:1, 1996.

A MMP-25 variant gene can be identified on the basis of structure by determining the level of identity with nucleotide or amino acid sequences of SEQ ID NO:1, 3 or 5 or SEQ ID NO:2, 4, or 6 as discussed above. An alternative approach to identifying a variant gene on the basis of structure is to determine whether a nucleic acid molecule encoding a potential variant MMP-25 gene can hybridize under normal or stringent conditions to a nucleic acid molecule having the nucleotide sequence of SEQ ID NO:1, 3 or 5 or to a fragment thereof of at least 15, 18, 24, 30, 50 or more nucleotides in length. As an illustration of moderate hybridization conditions, a nucleic acid molecule having a variant MMP-25 sequence can bind with a fragment of a nucleic acid molecule having a sequence from SEQ ID NO:1 in a buffer containing, for example, 5xSSPE (1XSSPE=180 mM sodium chloride, 10 mM sodium phosphate, 1 mM EDTA (pH 7.7), 5xDenhardt's solution (100xDenhardt's=2% (w/v) bovine serum albumin, 2% (w/v) Ficoll, 2% (w/v) polyvinylpyrrolidone) and 0.5% SDS incubated overnight at 55-60° C. Post-hybridization washes at high stringency are typically performed in 0.5xSSC (1xSSC=150 mM sodium chloride, 15 mM trisodium citrate) or in 0.5xSSPE at 55-60° C. Stringent hybridization conditions typically hybridize 1-2x SSPE (or equivalent salt concentration) overnight at 48-65° C., with or without a strand denaturant such 50% formamide, followed by a wash in 0.1-0.2% SSC at about 65° C.

Vectors

The invention provides for recombinant nucleic acid vectors comprising the aforementioned MMP-25 nucleic acids and related sequences. In a typical embodiment, the vector is an expression vector containing a promoter operably linked to the MMP-25 nucleic acid sequence for use in expressing a MMP-25 RNA, polypeptide or fragment thereof. The vector may be selected from any type of vector depending on intended use and host cell type. These include plasmid vectors, phage vectors, herpes simplex viral vectors, adenoviral vectors, adenovirus-associated viral vectors and retroviral vectors.

To express a MMP-25 gene, a nucleic acid molecule encoding the polypeptide must be operably linked to regulatory sequences that control transcriptional expression in an expression vector and then introduced into a host cell. In addition to transcriptional regulatory sequences, such as promoters and enhancers, expression vectors can include translational regulatory sequences and a marker gene which is suitable for selection of cells that carry the expression vector.

Expression vectors that are suitable for production of a foreign protein in eukaryotic cells typically contain (1) prokaryotic DNA elements coding for a bacterial replication origin and an antibiotic resistance marker to provide for the growth and selection of the expression vector in a bacterial host; (2) eukaryotic DNA elements that control initiation of transcription, such as a promoter; and (3) DNA elements that control the processing of transcripts, such as a transcription termination/polyadenylation sequence.

MMP-25 nucleic acids of the present invention are preferably expressed in mammalian cells. Examples of mammalian host cells include African green monkey kidney cells (Vero; ATCC CRL 1587), human embryonic kidney cells (293-HEK; ATCC CRL 1573), baby hamster kidney cells (BHK-21; ATCC CRL 8544), canine kidney cells (MDCK; ATCC CCL 34), Chinese hamster ovary cells (CHO-KI; ATCC CCL61), rat pituitary cells (GH1; ATCC CCL82), HeLa S3 cells (ATCC CCL2.2), rat hepatoma cells (H-4-41-E; ATCC CRL 1548) SV40-transformed monkey kidney cells (COS-1; ATCC CRL 1650) and murine embryonic cells (NIH-3T3; ATCC CRL 1658).

For a mammalian host, the transcriptional and translational regulatory signals may be derived from viral sources, such as adenovirus, bovine papilloma virus, simian virus, or the like, in which the regulatory signals are associated with a particular gene which has a high level of expression. Suitable transcriptional and translational regulatory sequences also can be obtained from mammalian genes, such as actin, collagen, myosin, and metallothionein genes.

Transcriptional regulatory sequences include a promoter region sufficient to direct the initiation of RNA synthesis. Suitable eukaryotic promoters include, for example, the promoter of the mouse metallothionein I gene (Hamer et al., J. Molec. Appl. Genet. 1:273, 1982), the TK promoter of Herpes virus (McKnight, Cell 31:355, 1982), the SV40 early promoter (Benoist et al., Nature 290:304, 1981), the Rous sarcoma virus promoter (Gorman et al., Proc. Nat'l Acad. Sci. USA 79:6777, 1982), the cytomegalovirus promoter (Foecking et al., Gene 45:101, 1980), and the mouse mammary tumor virus promoter (see, generally, Etcheverry, “Expression of Engineered Proteins in Mammalian Cell Culture,” in Protein Engineering:Principles and Practice, Cleland et al. (eds.), pages 163-181 (John Wiley & Sons, Inc. 1996)).

Alternatively, a prokaryotic promoter, such as the bacteriophage T3 RNA polymerase promoter, can be used to control MMP-25 gene expression in mammalian cells if the prokaryotic promoter is regulated by a eukaryotic promoter (Zhou et al., Mol. Cell. Biol. 10:4529, 1990; Kaufman et al., Nucl. Acids Res. 19:4485, 1991).

MMP-25 genes may also be expressed in bacterial, yeast, insect, or plant cells. Suitable promoters that can be used to express MMP-25 polypeptides in a prokaryotic host are well-known to those of skill in the art and include promoters capable of recognizing the T4, T3, Sp6 and T7 polymerases, the P_{R} and P_{L} promoters of bacteriophage lambda, the trp, recA, heat shock, lacUV5, tac, Ipp-lacSpr, phoA, and lacZ promoters of E. coli, promoters of B. subtilis, the promoters of the bacteriophages of Bacillus, Streptomyces promoters, the int promoter of bacteriophage lambda, the bla promoter of pBR322, and the CAT promoter of the chloramphenicol acetyl transferase gene. See Glick, J. Ind. Microbiol. 1:277, 1987, Watson et al., Molecular Biology of the Gene, 4th Ed. (Benjamin Cummins 1987), and by Ausubel et al. (1995).

Preferred prokaryotic hosts include E. coli and B. subtilus. Suitable strains of E. coli include BL21(DE3), BL21(DE3)pLysS, BL21(DE3)pLysE, DHI, DH4I, DH5, DH51, DH5IF′, DH5IMCR, DH10B, DH10B/p3, DH11S, C600, HB101, JM101, JM105, JM109, JM110, K38, RR1, Y1088, Y1089, CSH18, ER1451, and ER1647 (see, for example, Brown (Ed.), Molecular Biology Labfax (Academic Press 1991)). Suitable strains of Bacillus subtilus include BR151, YB886, MI1119, MI120, and B170 (see, for example, Hardy, “Bacillus Cloning Methods,” in DNA Cloning:A Practical Approach, Glover (Ed.) (IRL Press 1985)).

Methods for expressing proteins in prokaryotic hosts are well-known to those of skill in the art (see, for example, Williams et al., “Expression of foreign proteins in E. coli using plasmid vectors and purification of specific polyclonal antibodies,” in DNA Cloning 2:Expression Systems, 2nd Edition, Glover et al. (eds.), page 15 (Oxford University Press 1995); Ward et al., “Genetic Manipulation and Expression of Antibodies,” in Monoclonal Antibodies:Principles and Applications, page 137 (Wiley-Liss, Inc. 1995); and Georgiou, “Expression of Proteins in Bacteria,” in Protein Engineering:Principles and Practice, Cleland et al. (eds.), page 101 (John Wiley & Sons, Inc. 1996)).

The baculovirus system provides an efficient means to introduce cloned MMP-25 genes into insect cells. Suitable expression vectors are based upon the Autographa californica multiple nuclear polyhedrosis virus (AcMNPV), and contain well-known promoters such as Drosophila heat shock protein (hsp) 70 promoter, Autographa californica nuclear polyhedrosis virus immediate-early gene promoter (ie-1) and the delayed early 39K promoter, baculovirus p10 promoter, and the Drosophila metallothionein promoter. Suitable insect host cells include cell lines derived from IPLB-Sf-21, a Spodoptera frugiperda pupal ovarian cell line, such as Sf9 (ATCC CRL 1711), Sf21AE, and Sf21 (Invitrogen Corporation; San Diego, Calif.), as well as Drosophila Schneider-2 cells. Established techniques for producing recombinant proteins in baculovirus systems are provided by Bailey et al., “Manipulation of Baculovirus Vectors,” in Methods in Molecular Biology, Volume 7:Gene Transfer and Expression Protocols, Murray (ed.), pages 147-168 (The Humana Press, Inc. 1991), by Patel et al., “The baculovirus expression system,” in DNA Cloning 2:Expression Systems, 2nd Edition, Glover et al. (eds.), pages 205-244 (Oxford University Press 1995), by Ausubel (1995) at pages 16-37 to 16-57, by Richardson (ed.), Baculovirus Expression Protocols (The Humana Press, Inc. 1995), and by Lucknow, “Insect Cell Expression Technology,” in Protein Engineering:Principles and Practice, Cleland et al. (eds.), pages 183-218 (John Wiley & Sons, Inc. 1996).

Promoters for expression in yeast include promoters from GAL1 (galactose), PGK (phosphoglycerate kinase), ADH (alcohol dehydrogenase), AOXl (alcohol oxidase), HIS4 (histidinol dehydrogenase), and the like. Many yeast cloning vectors have been designed and are readily available. These vectors include Ylp-based vectors, such as YIp5, YRp vectors, such as YRp17, YEp vectors such as YEp13 and YCp vectors, such as YCp19. One skilled in the art will appreciate that there are a wide variety of suitable vectors for expression in yeast cells.

Expression vectors can also be introduced into plant protoplasts, intact plant tissues, or isolated plant cells. General methods of culturing plant tissues are provided, for example, by Miki et al., “Procedures for Introducing Foreign DNA into Plants,” in Methods in Plant Molecular Biology and Biotechnology, Glick et al. (eds.), pages 67-88 (CRC Press, 1993).

An expression vector can be introduced into host cells using a variety of standard techniques including calcium phosphate transfection, liposome-mediated transfection, microprojectile-mediated delivery, electroporation, and the like. Preferably, the transfected cells are selected and propagated to provide recombinant host cells that comprise the expression vector stably integrated in the host cell genome. Techniques for introducing vectors into eukaryotic cells and techniques for selecting such stable transformants using a dominant selectable marker are described, for example, by Ausubel (1995) and by Murray (ed.), Gene Transfer and Expression Protocols (Humana Press 1991). Methods for introducing expression vectors into bacterial, yeast, insect, and plant cells are also provided by Ausubel (1995).

### POLYPEPTIDES

As discussed above, vectors provided by the present invention are useful for producing MMP-25 polypeptides by expressing the polypeptide from the vector and isolating it from a host cell containing the same. Therefore, another aspect of the invention includes methods of producing a MMP-25 polypeptide comprising the step of culturing a host cell containing one of the aforementioned vectors containing a promoter operably linked to the MMP-25 sequence, under conditions and for a time sufficient to produce the MMP-25 polypeptide. In a preferred practice, the method further includes the step of purifying said MMP-25 polypeptide.

Accordingly, the invention also provides for a polypeptide comprising an amino acid sequence selected from the group consisting of: (a) the amino acid sequence according to SEQ ID NO:2, SEQ ID NO:4 or SEQ ID NO:6; (b) an amino acid sequence having at least 50% identity to the polypeptide of (a) or (b); (c) a sequence encoding a functional fragment of the polypeptide of (a) or (b); and (d) an amino acid sequence encoded by a nucleic acid that hybridizes under conditions of normal stringency or high stringency to these nucleic acids. More preferred embodiments of these polypeptides include those having at least 50%, 60%, 70%, 80%, 90%, or 95% identity to the polypeptide according to SEQ ID NO:2, SEQ ID NO:4 or SEQ ID NO:6. In particular embodiments, identity is calculated according the MEGALIGN algorithm referred to above, using a gap penalty and gap length penalty each set at a value of 10.

General methods for expressing and recovering foreign protein produced by a mammalian cell system is provided by, for example, Etcheverry, “Expression of Engineered Proteins in Mammalian Cell Culture,” in Protein Engineering:Principles and Practice, Cleland et al. (eds.), pages 163 (Wiley-Liss, Inc. 1996). Standard techniques for recovering protein produced by a bacterial system is provided by, for example, Grisshammer et al., “Purification of over-produced proteins from E. coli cells,” in DNA Cloning 2:Expression Systems, 2nd Edition, Glover et aL (eds.), pages 59-92 (Oxford University Press 1995). Established methods for isolating recombinant proteins from a baculovirus system are described by Richardson (ed.), Baculovirus Expression Protocols (The Humana Press, Inc., 1995).

More generally, MMP-25 can be isolated by standard techniques, such as affinity chromatography, size exclusion chromatography, ion exchange chromatography, HPLC and the like. Additional variations in MMP-25 isolation and purification can be devised by those of skill in the art. For example, anti-MMP-25 antibodies, obtained as described below, can be used to isolate large quantities of protein by immunoaffinity purification.

In one practice, MMP-25 polypeptides may be obtained from a host cell expressing a recombinant nucleic acid that encodes a MMP-25 polypeptide or portion thereof. For example, using recombinant DNA methods, a MMP-25 polypeptide can be isolated by culturing suitable host and vector systems to produce a native MMP-25 polypeptide. Alternatively, a vector can be selected for fusing a first nucleic acid segment encoding a MMP-25 peptide in-frame to a second nucleic acid segment containing a non- MMP-25 sequence. In a typical practice, the non-MMP-25 segment comprises a peptide or polypeptide that facilities isolation of the fusion molecule by binding to an antibody or a chemical matrix that binds to the non-MMP-25 segment. One common example uses a vector that provides a sequence encoding a histidine-tagged peptide (HIS-tag) and sites for fusion to the N-terminus or C-terminus of the MMP-25 segment. (For example, pET vectors from InVitrogen Inc., (Carlsbad, Calif.) or pQE-30 from Qiagen Inc., Valencia, Calif.) Also see U.S. Pat. No. 4,851,341, and Hopp et al., Bio/Technology 6:1204, 1988. This permits purification of the fusion polypeptide by binding to a nickel-chelating matrix. Alternatively, other tags may be used, including FLAG and GST. The associated tag can optionally be removed in a further step to obtain the MMP-25 polypeptide without the tag. For example, His-tagged proteins are incubated with thrombin, resulting in cleavage of a recognition sequence between the tag and the MMP-25 segment.

In an alternative practice, a vector can be engineered to export MMP-25 from the host cell or to retain MMP-25 in a readily isolated fraction of the host cell, for example within inclusion bodies in prokaryotic hosts. When engineered for export, a supernatant from a culture of the host cell can be used to isolate the exported MMP-25 polypeptide. Typically, the MMP-25 polypeptides used for export in a mammalian cell will include the same export signal that naturally occur with the MMP-25 such as the leader peptide as indicated in FIGS. 2 and 3. Alternatively, export signals such as leader peptide domains from different exported proteins can be fused to a MMP-25 polypeptide to provide for export in particular cell types.

When expressed in prokaryotic cells, MMP-25 may be isolated from inclusion bodies by a variety of purification procedures. For example, a fraction containing inclusion bodies can be separated from a soluble fraction of disrupted host cells by centrifugation or filtration and the MMP-25-polypeptide can be extracted therefrom using detergents. Optional further purification steps may include binding a sample to MMP-25 antibody bound to a suitable support. In addition, anion or cation exchange resins, gel filtration or affinity, hydrophobic or reverse phase chromatography may be employed in order to purify the protein.

In another alternative, the MMP-25 polypeptide can be isolated from an animal cell such as breast or skin cells in which it is naturally expressed. MMP-25 polypeptides can be purified by any of one or more of the steps common used to purify metalloproteinases generally. In addition or alternatively, the MMP-25 can be excised from a polyacrylamide gel after electrophoresis and identification of the appropriate 54 KD band on the gel as described in Example 5.

Fusion Proteins

The discussion above of isolation of proteins is equally applicable to the isolation of fusion proteins containing a portion of a MMP-25 polypeptide fused to another protein. Fusion proteins are useful for several purposes, including the combining of two or more catalytic functions from separate polypeptide sources, and for raising antibodies to epitopes. For raising antibodies to epitopes, the fusion protein typically contains a peptide epitope of a MMP-25 of at least 8, 10, 15 or 20 amino acids fused to a protein that enhances an immune response to the epitope. A typical protein for this purpose is KLH. Therefore, another aspect of the present invention provides a non-naturally occurring fusion protein, comprising a first MMP-25 polypeptide segment comprised of at least 8 contiguous amino acids of a MMP-25 polypeptide or variant described above, fused in- frame to a second polypeptide segment. The second polypeptide segment may comprise another portion of the MMP-25 polypeptide that is not naturally adjacent to the first segment, or comprise sequences from a non MMP-25 polypeptide.

Manipulation, Mutation and Expression of Polypeptides

Although various genes (or portions thereof) have been provided herein, it should be understood that within the context of the present invention, reference to one or more of these genes includes derivatives of the genes that are substantially similar to the genes (and, where appropriate, the proteins (including peptides and polypeptides) that are encoded by the genes and their derivatives). As used herein, a nucleotide sequence is deemed to be “substantially similar” if: (a) the nucleotide sequence is derived from the coding region of the above-described genes and includes, for example, portions of the sequence or allelic variations of the sequences discussed above, or alternatively, encodes a molecule which inhibits the binding of MMP-25 to a member of the MMP-25 family, (b) the nucleotide sequence is capable of hybridization to nucleotide sequences of the present inventionunder moderate, or high stringency as mentioned above. (also see Sambrook et al., Molecular Cloning: A Laboratory Manual, 2nd ed., Cold Spring Harbor Laboratory Press, N.Y., 1989); or (c) the DNA sequences are degenerate as a result of the genetic code in relation to the DNA sequences defined in (a) or (b). Further, the nucleic acid molecule disclosed herein includes both complementary and non-complementary sequences, provided the sequences otherwise meet the criteria set forth herein.

The structure of the proteins encoded by the nucleic acid molecules described herein may be predicted from the primary translation products using the hydrophobicity plot function of, for example, P/C Gene or Intelligenetics Suite (Intelligenetics, Mountain View, Calif.), or according to the methods described by Kyte and Doolittle (J. Mol. Biol. 157:105-132, 1982).

Proteins of the present invention may be prepared in the form of acidic or basic salts, or in neutral form. In addition, individual amino acid residues may be modified by oxidation or reduction. Furthermore, various substitutions, deletions, or additions may be made to the amino acid or nucleic acid sequences, the net effect of which is to retain or further enhance or decrease the biological activity of the mutant or wild-type protein. Moreover, due to degeneracy in the genetic code, for example, there may be considerable variation in nucleotide sequences encoding the same amino acid sequence.

Proteins of the present invention may be constructed using a wide variety of techniques described herein. Further, mutations may be introduced at particular loci by synthesizing oligonucleotides containing a mutant sequence, flanked by restriction sites enabling ligation to fragments of the native sequence. Following ligation, the resulting reconstructed sequence encodes a derivative having the desired amino acid insertion, substitution, or deletion.

Alternatively, oligonucleotide-directed site-specific (or segment specific) mutagenesis procedures may be employed to provide an altered gene having particular codons altered according to the substitution, deletion, or insertion required. Exemplary methods of making the alterations set forth above are disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and Sambrook et al. (supra). Deletion or truncation derivatives of proteins (e.g., a soluble extracellular portion) may also be constructed by utilizing convenient restriction endonuclease sites adjacent to the desired deletion. Subsequent to restriction, overhangs may be filled in, and the DNA religated. Exemplary methods of making the alterations set forth above are disclosed by Sambrook et al. (Molecular Cloning: A Laboratory Manual, 2d Ed., Cold Spring Harbor Laboratory Press, 1989).

Mutations which are made in the nucleic acid molecules of the present invention preferably preserve the reading frame of the coding sequences. Furthermore, the mutations will preferably not create complementary regions that could hybridize to produce secondary mRNA structures, such as loops or hairpins, that would adversely affect translation of the mRNA. Although a mutation site may be predetermined, it is not necessary that the nature of the mutation per se be predetermined. For example, in order to select for optimal characteristics of mutants at a given site, random mutagenesis may be conducted at the target codon and the expressed mutants screened for indicative biological activity. Alternatively, mutations may be introduced at particular loci by synthesizing oligonucleotides containing a mutant sequence, flanked by restriction sites enabling ligation to fragments of the native sequence. Following ligation, the resulting reconstructed sequence encodes a derivative having the desired amino acid insertion, substitution, or deletion.

Nucleic acid molecules which encode proteins of the present invention may also be constructed utilizing techniques of PCR mutagenesis, chemical mutagenesis (Drinkwater and Klinedinst, PNAS 83:3402-3406, 1986), by forced nucleotide misincorporation (e.g., Liao and Wise Gene 88:107-111, 1990), or by use of randomly mutagenized oligonucleotides (Horwitz et al., Genome 3:112-117, 1989).

The present invention also provides for the manipulation and expression of the above described genes by culturing host cells containing a vector capable of expressing the above-described genes. Such vectors or vector constructs include either synthetic or cDNA-derived nucleic acid molecules encoding the desired protein, which are operably linked to suitable transcriptional or translational regulatory elements. Suitable regulatory elements may be derived from a variety of sources, including bacterial, fungal, viral, mammalian, insect, or plant genes. Selection of appropriate regulatory elements is dependent on the host cell chosen, and may be readily accomplished by one of ordinary skill in the art. Examples of regulatory elements include: a transcriptional promoter and enhancer or RNA polymerase binding sequence, a transcriptional terminator, and a ribosomal binding sequence, including a translation initiation signal.

Nucleic acid molecules that encode any of the proteins described above may be readily expressed by a wide variety of prokaryotic and eukaryotic host cells, including bacterial, mammalian, yeast or other fungi, viral, insect, or plant cells as described above.

Techniques for transforming fungi are well known in the literature, and have been described, for instance, by Beggs (ibid.), Hinnen et al. (Proc. Natl. Acad. Sci. USA 75:1929-1933, 1978), Yelton et al. (Proc. Natl. Acad. Sci. USA 81:1740-1747, 1984), and Russell (Nature 301:167-169, 1983). The genotype of the host cell may contain a genetic defect that is complemented by the selectable marker present on the expression vector. Choice of a particular host and selectable marker is well within the level of ordinary skill in the art.

Protocols for the transformation of yeast are also well known to those of ordinary skill in the art. For example, transformation may be readily accomplished either by preparation of spheroplasts of yeast with DNA (see Hinnen et al., PNAS USA 75:1929, 1978) or by treatment with alkaline salts such as LiCl (see Itoh et al., J. Bacteriology 153:163, 1983). Transformation of fungi may also be carried out using polyethylene glycol as described by Cullen et al. (Bio/Technology 5:369, 1987).

Viral vectors include those which comprise a promoter that directs the expression of an isolated nucleic acid molecule that encodes a desired protein as described above. A wide variety of promoters may be utilized within the context of the present invention, including for example, promoters such as MoMLV LTR, RSV LTR, Friend MuLV LTR, adenoviral promoter (Ohno et al., Science 265:781-784, 1994), neomycin phosphotransferase promoter/enhancer, late parvovirus promoter (Koering et al., Hum. Gene Therap. 5:457-463, 1994), Herpes TK promoter, SV40 promoter, metallothionein IIa gene enhancer/promoter, cytomegalovirus immediate early promoter, and the cytomegalovirus immediate late promoter. Within particularly preferred embodiments of the invention, the promoter is a tissue-specific promoter (see e.g., WO 91/02805; EP 0,415,731; and WO 90/07936). Representative examples of suitable tissue specific promoters include neural specific enolase promoter, platelet derived growth factor beta promoter, bone morphogenic protein promoter, human alphal-chimaerin promoter, synapsin I promoter and synapsin II promoter. In addition to the above-noted promoters, other viral-specific promoters (e.g., retroviral promoters (including those noted above, as well as others such as HIV promoters), hepatitis, herpes (e.g., EBV), and bacterial, fungal or parasitic (e.g., malarial) -specific promoters may be utilized in order to target a specific cell or tissue which is infected with a virus, bacteria, fungus or parasite.

Mammalian cells suitable for carrying out the present invention include, among others COS, CHO, SaOS, osteosarcomas, KS483, MG-63, primary osteoblasts, and human or mammalian bone marrow stroma. Mammalian expression vectors for use in carrying out the present invention will include a promoter capable of directing the transcription of a cloned gene or cDNA. Preferred promoters include viral promoters and cellular promoters. Bone specific promoters include the bone sialo-protein and the promoter for osteocalcin. Viral promoters include the cytomegalovirus immediate early promoter (Boshart et al., Cell 41:521-530, 1985), cytomegalovirus immediate late promoter, SV40 promoter (Subramani et al., Mol. Cell. Biol. 1:854-864, 1981), MMTV LTR, RSV LTR, metallothionein-1, adenovirus E1a. Cellular promoters include the mouse metallothionein-1 promoter (Palmiter et al., U.S. Pat. No. 4,579,821), a mouse V_{K} promoter (Bergman et al., Proc. Natl. Acad. Sci. USA 81:7041-7045, 1983; Grant et al., Nucl. Acids Res. 15:5496, 1987) and a mouse V_{H} promoter (Loh et al., Cell 33:85-93, 1983). The choice of promoter will depend, at least in part, upon the level of expression desired or the recipient cell line to be transfected.

Such expression vectors may also contain a set of RNA splice sites located downstream from the promoter and upstream from the DNA sequence encoding the peptide or protein of interest. Preferred RNA splice sites may be obtained from adenovirus and/or immunoglobulin genes. Also contained in the expression vectors is a polyadenylation signal located downstream of the coding sequence of interest. Suitable polyadenylation signals include the early or late polyadenylation signals from SV40 (Kaufman and Sharp, ibid.), the polyadenylation signal from the Adenovirus 5 E1B region and the human growth hormone gene terminator (DeNoto et al., Nuc. Acids Res. 9:3719-3730, 1981). The expression vectors may include a noncoding viral leader sequence, such as the Adenovirus 2 tripartite leader, located between the promoter and the RNA splice sites. Preferred vectors may also include enhancer sequences, such as the SV40 enhancer. Expression vectors may also include sequences encoding the adenovirus VA RNAs. Suitable expression vectors can be obtained from commercial sources (e.g., Stratagene, La Jolla, Calif.).

Vector constructs comprising cloned DNA sequences can be introduced into cultured mammalian cells by, for example, calcium phosphate-mediated transfection (Wigler et al., Cell 14:725, 1978; Corsaro and Pearson, Somatic Cell Genetics 7:603, 1981; Graham and Van der Eb, Virology 52:456, 1973), electroporation (Neumann et al., EMBO J 1:841-845, 1982), or DEAE-dextran mediated transfection (Ausubel et al. (eds.), Current Protocols in Molecular Biology, John Wiley and Sons, Inc., NY, 1987). To identify cells that have stably integrated the cloned DNA, a selectable marker is generally introduced into the cells along with the gene or cDNA of interest. Preferred selectable markers for use in cultured mammalian cells include genes that confer resistance to drugs, such as neomycin, hygromycin, and methotrexate. The selectable marker may be an amplifiable selectable marker. Preferred amplifiable selectable markers are the DHFR gene and the neomycin resistance gene. Selectable markers are reviewed by Thilly (Mammalian Cell Technology, Butterworth Publishers, Stoneham, Mass., which is incorporated herein by reference).

Mammalian cells containing a suitable vector are allowed to grow for a period of time, typically 1-2 days, to begin expressing the DNA sequence(s) of interest. Drug selection is then applied to select for growth of cells that are expressing the selectable marker in a stable fashion. For cells that have been transfected with an amplifiable, selectable marker the drug concentration may be increased in a stepwise manner to select for increased copy number of the cloned sequences, thereby increasing expression levels. Cells expressing the introduced sequences are selected and screened for production of the protein of interest in the desired form or at the desired level. Cells that satisfy these criteria can then be cloned and scaled up for production.

Protocols for the transfection of mammalian cells are well known to those of ordinary skill in the art. Representative methods include calcium phosphate mediated transfection, electroporation, lipofection, retroviral, adenoviral and protoplast fusion- mediated transfection (see Sambrook et al., supra). Naked vector constructs can also be taken up by muscular cells or other suitable cells subsequent to injection into the muscle of a mammal (or other animals).

Numerous insect host cells known in the art can also be useful within the present invention, in light of the subject specification. For example, the use of baculoviruses as vectors for expressing heterologous DNA sequences in insect cells has been reviewed by Atkinson et al. (Pestic. Sci. 28:215-224,1990).

Numerous plant host cells known in the art can also be useful within the present invention, in light of the subject specification. For example, the use of Agrobacterium rhizogenes as vectors for expressing genes in plant cells has been reviewed by Sinkar et al. (J. Biosci. (Bangalore) 11:47-58, 1987).

Within related aspects of the present invention, proteins of the present invention may be expressed in a transgenic animal whose germ cells and somatic cells contain a gene which encodes the desired protein and which is operably linked to a promoter effective for the expression of the gene. Alternatively, in a similar marner transgenic animals may be prepared that lack the desired gene (e.g., “knock-out” mice). Such transgenics may be prepared in a variety of non-human animals, including mice, rats, rabbits, sheep, dogs, goats and pigs (see Hammer et al., Nature 315:680-683, 1985, Palmiter et al., Science 222:809-814, 1983, Brinster et al., Proc. Natl. Acad. Sci. USA 82:4438-4442, 1985, Palmiter and Brinster, Cell 41:343-345, 1985, and U.S. Pat. Nos. 5,175,383, 5,087,571, 4,736,866, 5,387,742, 5,347,075, 5,221,778, and 5,175,384). Briefly, an expression vector, including a nucleic acid molecule to be expressed together with appropriately positioned expression control sequences, is introduced into pronuclei of fertilized eggs, for example, by microinjection. Integration of the injected DNA is detected by blot analysis of DNA from tissue samples. It is preferred that the introduced DNA be incorporated into the germ line of the animal so that it is passed on to the animal's progeny. Tissue-specific expression may be achieved through the use of a tissue-specific promoter, or through the use of an inducible promoter, such as the metallothionein gene promoter (Palmiter et al., 1983, ibid), which allows regulated expression of the transgene.

### ANTIBODIES

The polypeptides of the present invention are useful for raising antibodies which bind specifically or preferentially to MMP-25 polypeptides. Accordingly, another aspect of the invention provides an antibody that binds to a MMP, wherein said antibody specifically binds to at least one polypeptide or peptide fragment according to SEQ ID NOS:2, 4, or 6, or to variants thereof as discussed above. In one embodiment, the antibody is a monoclonal antibody. Typically the antibody will bind to a type 25 MMP with a higher affinity than it binds to a non type 25 MMP. The antibody is also typically, a murine or human antibody. Related aspects of the antibodies of the present invention include an antibody selected from the group consisting of F(ab′)₂, F(ab)₂, Fab′ Fab and Fv, and a hybridoma which produces the aforementioned monoclonal antibody.

Antibodies to MMP-25 polypeptides are useful in another aspect of the invention, which is to identify or isolate MMP-25 polypeptide and peptide sequences. Therefore, the invention also includes a method of identifying a type 25 MMP polypeptide comprising incubating an antibody that specifically binds with a MMP-25 polypeptide with a sample containing protein for a time sufficient to permit said antibody to bind the type 25 MMP present in the sample. In a typical practice of this method, the antibody is bound to a solid support and optionally labeled to facilitate its detection.

Antibodies to MMP-25 can be obtained, for example, using the product of an expression vector as an antigen. Particularly useful anti-MMP-25 antibodies “bind specifically” with MMP-25 polypeptides of SEQ ID NOs. 2, 4 or 6 and variants thereof in that they bind to the MMP-25 polypeptide with a higher affinity than to a non-type 25 MMP protein such as MMP 1-3 or 7-22. Antibodies of the present invention (including fragments and derivatives thereof) may be a polyclonal, or especially, a monoclonal antibody. The antibody may belong to any immunoglobulin class, and may be for example an IgG, for example IgG₁, IgG₂, IgG₃, IgG₄; IgE; IgM; or IgA antibody. It may be of animal, for example mammalian origin, and may be for example a murine, rat, human or other primate antibody.

Polyclonal antibodies to recombinant MMP-25 can be prepared using methods well-known to those of skill in the art (see, for example, Green et al., “Production of Polyclonal Antisera,” in Immunochemical Protocols (Manson, ed.), pages 1-5 (Humana Press 1992); Williams et al., “Expression of foreign proteins in E. coli using plasmid vectors and purification of specific polyclonal antibodies,” in DNA Cloning 2:Expression Systems, 2nd Edition, Glover et al. (eds.), page 15 (Oxford University Press 1995)). Although polyclonal antibodies are typically raised in animals such as rats, mice, rabbits, goats, or sheep, an anti-MMP-25 antibody of the present invention may also be derived from a subhuman primate antibody. General techniques for raising diagnostically and therapeutically useful antibodies in baboons may be found, for example, in Goldenberg et al., International Patent publication No. WO 91/11465 (1991), and in Losman et al., Int. J. Cancer 46:310, 1990.

Briefly, polyclonal antibodies may be readily generated by one of ordinary skill in the art from a variety of warm-blooded animals such as horses, cows, various fowl, rabbits, mice, or rats. Typically, the MMP-25 or unique peptide thereof of 13-20 amino acids (preferably conjugated to keyhole limpet hemocyanin by cross-linking with glutaraldehyde) is utilized to immunize the animal through intraperitoneal, intramuscular, intraocular, or subcutaneous injections, along with an adjuvant such as Freund's complete or incomplete adjuvant. Following several booster immunizations, samples of serum are collected and tested for reactivity to the protein or peptide. Particularly preferred polyclonal antisera will give a signal on one of these assays that is at least three times greater than background. Once the titer of the animal has reached a plateau in terms of its reactivity to the protein, larger quantities of antisera may be readily obtained either by weekly bleedings, or by exsanguinating the animal.

The antibody should comprise at least a variable region domain. The variable region domain may be of any size or amino acid composition and will generally comprise at least one hypervariable amino acid sequence responsible for antigen binding embedded in a framework sequence. In general terms the variable (V) region domain may be any suitable arrangement of immunoglobulin heavy (V_{H}) and/or light (V_{L}) chain variable domains. Thus for example the V region domain may be monomeric and be a V_{H} or V_{L} domain where these are capable of independently binding antigen with acceptable affinity. Alternatively the V region domain may be dimeric and contain V_{H}-V_{H}, V_{H}-V_{L}, or V_{L}-V_{L}, dimers in which the V_{H} and V_{L} chains are non-covalently associated (abbreviated hereinafter as Fᵥ). Where desired, however, the chains may be covalently coupled either directly, for example via a disulphide bond between the two variable domains, or through a linker, for example a peptide linker, to form a single chain domain (abbreviated hereinafter as scFᵥ).

The variable region domain may be any naturally occurring variable domain or an engineered version thereof. By engineered version is meant a variable region domain which has been created using recombinant DNA engineering techniques. Such engineered versions include those created for example from natural antibody variable regions by insertions, deletions or changes in or to the amino acid sequences of the natural antibodies. Particular examples of this type include those engineered variable region domains containing at least one CDR and optionally one or more framework amino acids from one antibody and the remainder of the variable region domain from a second antibody.

The variable region domain may be covalently attached at a C-terminal amino acid to at least one other antibody domain or a fragment thereof. Thus, for example where a V_{H} domain is present in the variable region domain this may be linked to an immunoglobulin C_{H}1 domain or a fragment thereof. Similarly a V_{L} domain may be linked to a C_{K} domain or a fragment thereof. In this way for example the antibody may be a Fab fragment wherein the antigen binding domain contains associated V_{H} and V_{L} domains covalently linked at their C-termini to a CH1 and C_{K} domain respectively. The CH1 domain may be extended with further amino acids, for example to provide a hinge region domain as found in a Fab′ fragment, or to provide further domains, such as antibody CH2 and CH3 domains.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (see, for example, Larrick et al., Methods:A Companion to Methods in Enzymology 2:106, 1991; Courtenay-Luck, “Genetic Manipulation of Monoclonal Antibodies,” in Monoclonal Antibodies:Production, Engineering and Clinical Application, Ritter et al. (eds.), page 166 (Cambridge University Press 1995); and Ward et al., “Genetic Manipulation and Expression of Antibodies,” in Monoclonal Antibodies:Principles and Applications, Birch et al. (eds.), page 137 (Wiley-Liss, Inc. 1995)).

Antibodies for use in the invention may in general be monoclonal prepared by conventional immunisation and cell fusion procedures) or in the case of fragments, derived therefrom using any suitable standard chemical e.g., reduction or enzymatic cleavage and/or digestion techniques, for example by treatment with pepsin.

More specifically, monoclonal anti-MMP-25 antibodies can be generated utilizing a variety of techniques. Rodent monoclonal antibodies to specific antigens may be obtained by methods known to those skilled in the art (see, for example, Kohler et al., Nature 256:495, 1975; and Coligan et al. (eds.), Current Protocols in Immunology, 1:2.5.1-2.6.7 (John Wiley & Sons 1991) [“Coligan”]; Picksley et al., “Production of monoclonal antibodies against proteins expressed in E. coli,” in DNA Cloning 2:Expression Systems, 2nd Edition, Glover et al. (eds.), page 93 (Oxford University Press 1995)).

The affinity of a monoclonal antibody or binding partner, as well as inhibition of binding can be readily determined by one of ordinary skill in the art (see Scatchard, Ann. N.Y. Acad. Sci. 51:660-672, 1949).

Monoclonal antibodies may also be readily generated using techniques described for example, U.S. Pat. Nos. 4,902,614, 4,543,439, and 4,411,993 which are incorporated herein by reference; see also Monoclonal Antibodies, Hybridomas: A New Dimension in Biological Analyses, Plenum Press, Kennett, McKeam, and Bechtol (eds.), 1980, and Antibodies:A Laboratory Manual, Harlow and Lane (eds.), Cold Spring Harbor Laboratory Press, 1988, which are also incorporated herein by reference).

Within one example practice, a subject animal such as a rat or mouse is immunized with MMP-25 or portion thereof as described above. The protein may be admixed with an adjuvant such as Freund's complete or incomplete adjuvant in order to increase the resultant immune response. Between one and three weeks after the initial immunization the animal may be reimmunized with another booster immunization, and tested for reactivity to the protein utilizing assays described above. Once the animal has reached a plateau in its reactivity to the injected protein, it is sacrificed, and organs which contain large numbers of B cells such as the spleen and lymph nodes are harvested.

Cells which are obtained from the immunized animal may be immortalized by infection with a virus such as the Epstein-Barr virus (EBV) (see Glasky and Reading, Hybridoma 8(4):377-389, 1989). Alternatively, within a preferred embodiment, the harvested spleen and/or lymph node cell suspensions are fused with a suitable myeloma cell in order to create a “hybridoma” which secretes monoclonal antibody. Suitable myeloma lines include, for example, NS-1 (ATCC No. TIB 18), and P3X63 - Ag 8.653 (ATCC No. CRL 1580).

Following the fusion, the cells may be placed into culture plates containing a suitable medium, such as RPMI 1640, or DMEM (Dulbecco's Modified Eagles Medium) (JRH Biosciences, Lenexa, Kans.), as well as additional ingredients, such as fetal bovine serum (FBS, i.e., from Hyclone, Logan, Utah, or JRH Biosciences). Additionally, the medium should contain a reagent which selectively allows for the growth of fused spleen and myeloma cells such as HAT (hypoxanthine, aminopterin, and thymidine) (Sigma Chemical Co., St. Louis, Mo.). After about seven days, the resulting fused cells or hybridomas may be screened in order to determine the presence of antibodies which are reactive against MMP-25 (depending on the antigen used), and which block or inhibit the binding of MMP-25 to a MMP-25 family member.

A wide variety of assays may be utilized to determine the presence of antibodies which are reactive against the proteins of the present invention, including for example countercurrent immuno-electrophoresis, radioimmunoassays, radioimmunoprecipitations, enzyme-linked immunosorbent assays (ELISA), dot blot assays, western blots, immunoprecipitation, inhibition or competition assays, and sandwich assays (see U.S. Pat. Nos. 4,376,110 and 4,486,530; see also Antibodies: A Laboratory Manual, Harlow and Lane (eds.), Cold Spring Harbor Laboratory Press, 1988). Following several clonal dilutions and reassays, a hybridoma producing antibodies reactive against the desired protein may be isolated.

Still other techniques may also be utilized to construct monoclonal antibodies (see William D. Huse et al., “Generation of a Large Combinational Library of the Immunoglobulin Repertoire in Phage Lambda,” Science 246:1275-1281, December 1989; see also L. Sastry et al., “Cloning of the Immunological Repertoire in Escherichia coli for Generation of Monoclonal Catalytic Antibodies: Construction of a Heavy Chain Variable Region-Specific cDNA Library,” Proc. Natl. Acad. Sci. USA 86:5728-5732, August 1989; see also Michelle Alting-Mees et al., “Monoclonal Antibody Expression Libraries: A Rapid Alternative to Hybridomas,” Strategies in Molecular Biology 3:1-9, January 1990). These references describe a commercial system available from Stratagene (La Jolla, Calif.) which enables the production of antibodies through recombinant techniques. Briefly, mRNA is isolated from a B cell population, and utilized to create heavy and light chain immunoglobulin CDNA expression libraries in the λlmmunoZap(H) and λlmmunoZap(L) vectors. These vectors may be screened individually or co-expressed to form Fab fragments or antibodies (see Huse et al., supra; see also Sastry et al., supra). Positive plaques may subsequently be converted to a non-lytic plasmid which allows high level expression of monoclonal antibody fragments from E. coli.

Similarly, portions or fragments, such as Fab and Fv fragments, of antibodies may also be constructed utilizing conventional enzymatic digestion or recombinant DNA techniques to incorporate the variable regions of a gene which encodes a specifically binding antibody. Within one embodiment, the genes which encode the variable region from a hybridoma producing a monoclonal antibody of interest are amplified using nucleotide primers for the variable region. These primers may be synthesized by one of ordinary skill in the art, or may be purchased from commercially available sources. Stratagene (La Jolla, Calif.) sells primers for mouse and human variable regions including, among others, primers for V_{Ha}, V_{Hb}, V_{HC}, V_{Hd}, C_{HI}, V_{L} and C_{L} regions. These primers may be utilized to amplify heavy or light chain variable regions, which may then be inserted into vectors such as ImmunoZAP^{™} H or ImmunoZAP^{™} L (Stratagene), respectively. These vectors may then be introduced into E. coli, yeast, or mammalian-based systems for expression. Utilizing these techniques, large amounts of a single-chain protein containing a fusion of the V_{H} and V_{L} domains may be produced (see Bird et al., Science 242:423-426, 1988). In addition, such techniques may be utilized to change a “murine” antibody to a “human” antibody, without altering the binding specificity of the antibody.

Once suitable antibodies have been obtained, they may be isolated or purified by many techniques well known to those of ordinary skill in the art (see Antibodies:A Laboratory Manual, Harlow and Lane (eds.), Cold Spring Harbor Laboratory Press, 1988). Suitable techniques include peptide or protein affinity columns, HPLC or RP-HPLC, purification on protein A or protein G columns, or any combination of these techniques.

In addition, an anti-MMP-25 antibody of the present invention may be derived from a human monoclonal antibody. Human monoclonal antibodies are obtained from transgenic mice that have been engineered to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described, for example, by Green et al., Nature Genet. 7:13, 1994; Lonberg et al., Nature 368:856, 1994; and Taylor et al., Int. Immun. 6:579, 1994.

Monoclonal antibodies can be isolated and purified from hybridoma cultures by a variety of well-established techniques. Such isolation techniques include affinity chromatography with Protein-A Sepharose, size-exclusion chromatography, and ion-exchange chromatography (see, for example, Coligan at pages 2.7.1-2.7.12 and pages 2.9.1-2.9.3; Baines et al., “Purification of Immunoglobulin G (IgG),” in Methods in Molecular Biology, Vol. 10, pages 79-104 (The Humana Press, Inc. 1992)).

For particular uses, it may be desirable to prepare fragments of anti-MMP-25 antibodies. Such antibody fragments can be obtained, for example, by proteolytic hydrolysis of the antibody. Antibody fragments can be obtained by pepsin or papain digestion of whole antibodies by conventional methods. As an illustration, antibody fragments can be produced by enzymatic cleavage of antibodies with pepsin to provide a 5S fragment denoted F(ab′)₂. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using pepsin produces two monovalent Fab fragments and an Fc fragment directly. These methods are described, for example, by Goldenberg, U.S. Pat. No. 4,331,647, Nisonoff et al., Arch Biochem. Biophys. 89:230, 1960, Porter, Biochem. J 73:119, 1959, Edelman et al., in Methods in Enzymology 1:422 (Academic Press 1967), and by Coligan at pages 2.8.1-2.8.10 and 2.10.-2.10.4.

Other methods of cleaving antibodies, such as separation of heavy chains to form monovalent light-heavy chain fragments, further cleavage of fragments, or other enzymatic, chemical or genetic techniques may also be used, so long as the fragments bind to the antigen that is recognized by the intact antibody.

Alternatively, the antibody may be a recombinant or engineered antibody obtained by the use of recombinant DNA techniques involving the manipulation and re- expression of DNA encoding antibody variable and/or constant regions. Such DNA is known and/or is readily available from DNA libraries including for example phage-antibody libraries (see Chiswell, D J and McCafferty, J. Tibtech. 10 80-84 (1992)) or where desired can be synthesised. Standard molecular biology and/or chemistry procedures may be used to sequence and manipulate the DNA, for example, to introduce codons to create cysteine residues, to modify, add or delete other amino acids or domains as desired.

In this practice, one or more replicable expression vectors containing the DNA may be prepared and used to transform an appropriate cell line, e.g., a non-producing myeloma cell line, such as a mouse NSO line or a bacterial, e.g., E. coli line, in which production of the antibody will occur. In order to obtain efficient transcription and translation, the DNA sequence in each vector should include appropriate regulatory sequences, particularly a promoter and leader sequence operably linked to the variable domain sequence. Particular methods for producing antibodies in this way are generally well known and routinely used. For example, basic molecular biology procedures are described by Maniatis et al (Molecular Cloning, Cold Spring Harbor Laboratory, New York, 1989); DNA sequencing can be performed as described in Sanger et al. (PNAS 74:5463 (1977)) and the Amersham International plc sequencing handbook; and site directed mutagenesis can be carried out according to the method of Kramer et al. (Nucl. Acids Res. 12:9441 (1984)) and the Anglian Biotechnology Ltd handbook. Additionally, there are numerous publications, detailing techniques suitable for the preparation of antibodies by manipulation of DNA, creation of expression vectors and transformation of appropriate cells, for example as reviewed by Mountain A and Adair, J R in Biotechnology and Genetic Engineering Reviews (ed. Tombs, M P, 10, Chapter 1, 1992, Intercept, Andover, UK) and in International Patent Specification No. WO 91/09967.

Where desired, the antibody according to the invention may have one or more effector or reporter molecules attached to it and the invention extends to such modified proteins. The effector or reporter molecules may be attached to the antibody through any available amino acid side-chain, terminal amino acid or, where present carbohydrate functional group located in the antibody, always provided of course that this does not adversely affect the binding properties and eventual usefulness of the molecule. Particular functional groups include, for example any free amino, imino, thiol, hydroxyl, carboxyl or aldehyde group. Attachment of the antibody and the effector and/or reporter molecule(s) may be achieved via such groups and an appropriate functional group in the effector or reporter molecules. The linkage may be direct or indirect, through spacing or bridging groups.

Effector molecules include, for example, antineoplastic agents, toxins (such as enzymatically active toxins of bacterial or plant origin and fragments thereof e.g., ricin and fragments thereof) biologically active proteins, for example enzymes, nucleic acids and fragments thereof, e.g., DNA, RNA and fragments thereof, naturally occurring and synthetic polymers e.g., polysaccharides and polyalkylene polymers such as poly(ethylene glycol) and derivatives thereof, radionuclides, particularly radioiodide, and chelated metals. Suitable reporter groups include chelated metals, fluorescent compounds or compounds which may be detected by NMR or ESR spectroscopy.

Particular antineoplastic agents include cytotoxic and cytostatic agents, for example alkylating agents, such as nitrogen mustards (e.g., chlorambucil, melphalan, mechlorethamine, cyclophosphamide, or uracil mustard) and derivatives thereof, triethylenephosphoramide, triethylenethiophosphor-amide, busulphan, or cisplatin; antimetabolites, such as methotrexate, fluorouracil, floxuridine, cytarabine, mercaptopurine, thioguanine, fluoroacetic acid or fluorocitric acid, antibiotics, such as bleomycins (e.g., bleomycin sulphate), doxorubicin, daunorubicin, mitomycins (e.g., mitomycin C), actinomycins (e.g., dactinomycin) plicamycin, calichaemicin and derivatives thereof, or esperamicin and derivatives thereof; mitotic inhibitors, such as etoposide, vincristine or vinblastine and derivatives thereof; alkaloids, such as ellipticine; polyols such as taxicin-I or taxicin-II; hormones, such as androgens (e.g., dromostanolone or testolactone), progestins (e.g., megestrol acetate or medroxyprogesterone acetate), estrogens (e.g., dimethylstilbestrol diphosphate, polyestradiol phosphate or estramustine phosphate) or antiestrogens (e.g., tamoxifen); anthraquinones, such as mitoxantrone, ureas, such as hydroxyurea; hydrazines, such as procarbazine; or imidazoles, such as dacarbazine.

Particularly useful effector groups are calichaemicin and derivatives thereof (see for example South African Patent Specifications NOS. 85/8794, 88/8127 and 90/2839).

Chelated metals include chelates of di-or tripositive metals having a coordination number from 2 to 8 inclusive. Particular examples of such metals include technetium (Tc), rhenium (Re), cobalt (Co), copper (Cu), gold (Au), silver (Ag), lead (Pb), bismuth (Bi), indium (In), gallium (Ga), yttrium (Y), terbium (Th), gadolinium (Gd), and scandium (Sc). In general the metal is preferably a radionuclide. Particular radionuclides include ^{99m}Tc, ¹⁸⁶Re, ¹⁸⁸Re, ⁵⁸Co, ⁶⁰Co, ⁶⁷Cu, ¹⁹⁵Au, ¹⁹⁹Au, ¹¹⁰Ag, ²⁰³Pb, ²⁰⁶Bi, ²⁰⁷Bi, ¹¹¹In, ⁶⁷Ga, ⁶⁸Ga, ⁸⁸Y, ⁹⁰Y, ¹⁶⁰Tb, ¹⁵³Gd and ⁴⁷Sc.

The chelated metal may be for example one of the above types of metal chelated with any suitable polydentate chelating agent, for example acyclic or cyclic polyamines, polyethers (e.g., crown ethers and derivatives thereof); polyamides; porphyrins; and carbocyclic derivatives.

In general, the type of chelating agent will depend on the metal in use. One particularly useful group of chelating agents in conjugates according to the invention, however, are acyclic and cyclic polyamines, especially polyaminocarboxylic acids, for example diethylenetriaminepentaacetic acid and derivatives thereof, and macrocyclic amines, e.g., cyclic tri-aza and tetra-aza derivatives (for example as described in International Patent Specification No. WO 92/22583); and polyamides, especially desferrioxamine and derivatives thereof.

Thus for example, when it is desired to use a thiol group in the antibody as the point of attachment this may be achieved through reaction with a thiol reactive group present in the effector or reporter molecule. Examples of such groups include an a-halocarboxylic acid or ester, e.g., iodoacetamide, an imide, e.g., maleimide, a vinyl sulphone, or a disulphide. These and other suitable linking procedures are generally and more particularly described in International Patent Specifications NOS. WO 93/06231, WO 92/22583, WO 90/091195 and WO 89/01476.

### RIBOZYMES AND ANTISENSE MOLECULES

In another aspect, the nucleic acid sequences of the present invention provide for nucleic acids useful for modulating or inhibiting the expression of a MMP-25 polypeptide in a cell. More specifically, the invention provides for a ribozyme that cleaves RNA encoding the aforementioned MMP-25 polypeptides. Also included is a nucleic acid molecule comprising a sequence that encodes such a ribozyme and a vector comprising the nucleic acid molecule. In a similar aspect, the invention provides antisense nucleic acid molecule comprising a sequence that is antisense to a portion of the MMP-25 nucleic acids described herein. Also included are a vector comprising the antisense molecule, and vectors wherein the aforementioned ribozyme or antisense nucleic acid is operably linked to a promoter. Typical embodiments of these vectors are selected from the group consisting of plasmid vectors, phage vectors, herpes simplex viral vectors, adenoviral vectors, adenovirus-associated viral vectors and retroviral vectors. Host cells comprising the above vectors are also included.

Antisense oligonucleotide molecules are provided which specifically inhibit expression of MMP-25 nucleic acid sequences (see generally, Hirashima et al. in Molecular Biology of RNA:New Perspectives (M. Inouye and B. S. Dudock, eds., 1987 Academic Press, San Diego, p. 401); Oligonucleotides:Antisense Inhibitors of Gene Expression (J. S. Cohen, ed., 1989 MacMillan Press, London); Stein and Cheng, Science 261:1004-1012, 1993; WO 95/10607; U.S. Pat. No. 5,359,051; WO 92/06693; and EP-A2-612844). Briefly, such molecules are constructed such that they are complementary to, and able to form Watson-Crick base pairs with, a region of transcribed MMP-25 mRNA sequence. The resultant double-stranded nucleic acid interferes with subsequent processing of the mRNA, thereby preventing protein synthesis (Example 6).

Ribozymes are provided which are capable of inhibiting expression of MMP-25 RNA. As used herein, “ribozymes” are intended to include RNA molecules that contain anti-sense sequences for specific recognition, and an RNA-cleaving enzymatic activity. The catalytic strand cleaves a specific site in a target RNA at greater than stoichiometric concentration. A wide variety of ribozymes may be utilized within the context of the present invention, including for example, the hammerhead ribozyme (for example, as described by Forster and Symons, Cell 48:211-220, 1987; Haseloff and Gerlach, Nature 328:596-600, 1988; Walbot and Bruening, Nature 334:196, 1988; Haseloff and Gerlach, Nature 334:585, 1988); the hairpin ribozyme (for example, as described by Haseloffet al., U.S. Pat. No.5,254,678, issued Oct. 19, 1993 and Hempel et al., European Patent Publication No. 0 360 257, published Mar. 26, 1990); and Tetrahymena ribosomal RNA-based ribozymes (see Cech et al., U.S. Pat. No. 4,987,071). Ribozymes of the present invention typically consist of RNA, but may also be composed of DNA, nucleic acid analogs (e.g., phosphorothioates), or chimerics thereof (e.g., DNA/RNA/RNA).

### METHODS OF INHIBITING MMP-25 ACTIVITY

MMP sequences lacking a Zn/Ca-binding domain

As noted above, the MMP-25(s) sequence differs from the MMP-25(l) sequence in that it lacks a portion of the second Zn/Ca-binding domain. While not being bound by theory, one explanation is that MMP-25(s) represents a non-functional splice variant of the longer sequence. Expression of a non-functional variant of a matrix metalloproteinase in the same cells that express a non-functional variant is one mechanism for regulating overall matrix metalloproteinase activity. For example, Rubins et al. (U.S. Pat. No. 5,935,792) discloses that expression of a non-functional variant of KUZ family MMP during neurogenesis of Drosophila cells interferes with the activity of a functional KUZ variant, thereby acting as a dominant negative regulator of MMP activity. Perturbation of this dominant negative regulation in Drosophila cells in turn perturbs neurogenesis resulting in the overproduction of primary neurons.

The MMP-25(s) sequence provided by the present invention may serve an analogous role in the regulation of other MMPs expressed in the same cell (e.g., MMP-25(l)). This provides a useful mechanism for manipulation of overall MMP activity in these cells by modulating the expression of MMP-25(s). More generally, the expression of a MMP lacking a means for Zn/Ca-binding domain is one particular method of inhibiting the overall MMP activity in the cell, including the activity provided by a similar sequence that does contain the Zn/Ca-binding domain.

Another explanation for the presence of MMP-25(s) is that it provides for a novel type of MMP catalytic activity. The previously observed consensus of two Zn-binding domains in all MMP proteins has lead to the speculation that both binding domains are required for MMP catalytic activity. Accordingly, MMP-25(s) and MMP-25(l) may represent MMPs having alternative types of catalytic activity, i.e., a first MMP activity conveyed by means of two Zn-binding domains, and a second MMP activity conveyed by means of a single Zn-binding domain. This discovery would provide a method for altering the catalytic activity of any MMPs by deleting or substituting the means conveyed by the second Zn/Ca-binding domain and retaining only means conveyed by the first Zn-binding domain.

Therefore, another aspect of the present invention provides a MMP sequence that has only one Zn-binding domain rather than the two normally associated with a MMP. More specifically, the invention provides for a polypeptide comprising a MMP of at least 471 amino acid residues in length, where the polypeptide is comprised of a first MMP Zn-binding domain and with the proviso that the polypeptide lacks a second MMP Zn-binding domain (the Zn²⁺/Ca²⁺ binding domain). In certain embodiments, the polypeptide may exhibit a catalytic activity of a MMP providing for a novel type of enzymatic activity. In another embodiment, the polypeptide will be non-functional and lack a catalytic activity, making it useful for down regulating overall MMP activity when expressed in the same cell. Catalytic activity can be readily assessed by methods known in the art for measuring MMP activity of a particular MMP, for example, by the ghost band procedure described in Example 5.

Inhibiting catalytic activity of a MMP-25

In a more general aspect, the MMP-25 sequences of the present invention provide protein targets for inhibiting MMP catalytic activity. More specifically, the invention provides a method of inhibiting a catalytic activity of a MMP polypeptide in a cell, comprising administering an agent to the cell that inhibits a catalytic activity of the MMP, with the proviso that the agent inhibits the catalytic activity of a MMP-25 polypeptide to a greater extent than it inhibits the activity of at least one non-type 25 MMP. In a preferred practice of this method, the MMP-25 polypeptide is preferentially expressed in the cell relative to the non-type 25 MMP. In one embodiment, the agent is topically administered to a skin cell of an animal.

Example MMP inhibitor agents for use in this method include:1,10-phenanthroline (o-phenanthroline); batimastat also known as BB-94; 4-(N-hydroxyamino)-2R-isobutyl-3S-(thiopen-2-ylthiomethyl)-succinyl-L-phenylalanine-N-methylamidecarboxyalkylamino-based compounds such as N-i-(R)-carboxy-3-(1,3-dihydro-2H-benzfisoindol-2-yl)propyl-N′,N′-dime thyl-L-leucinamide, trifluoroacetate (J. Med Chem. 36:4030-4039, 1993); marimastat (BB-2516); N-chlorotaurine; eicosapentaenoic acid; matlystatin-B; actinonin (3-1-2-(hydroxymethyl)-1-pyrolidinylcarbamoyl-octanohydroxamic acid); N-phosphonalkyl dipeptides such as N-N-((R)-1-phosphonopropyl)-(S)-leucyl-(S)-phenylalanine-N-methylamide (J. Med. Chem. 37:158-169, 1994); peptidyl hydroxamic acids such as pNH.sub.2 -Bz-Gly-Pro-D-Leu-D-Ala-NHOH (Biophys. Biochem. Res. Comm. 199:1442-1446, 1994); Ro-31-7467, also known as 2-(5-bromo-2,3-dihydro-6-hydroxy-1,3-dioxo-1 H benzdelisoquinolin-2-yl)met hyl(hydroxy)-phosphinyl-N-(2-oxo-3-azacyclotridecanyl)-4-methylvaleramid e; CT 1166, also known as N1N2-(morpholinosulphonylamino)-ethyl-3-cyclohexyl-2-(S)-propanamidyl -N4-hydroxy-2-(R)-3-(4-methylphenyl)propyl-succinamide (Biochem. J 308:167-175, 1995); bromocyclic-adenosine monophosphate; protocatechuic aldehyde (3,4-dihydroxybenzaldehyde); estramustine (estradiol-3-bis(2-chloroethyl)carbamate); tetracycline (4-(dimethylamino)- 1,4,4a,5,5a,6,11,12a-octahydro-3,6,10,12,12a-pentahydro xy-6-methyl-1,11-dioxo-2-naphthacenecarboxamide); minocycline (7-dimethylamino-6-dimethyl-6-deoxytetracycline); methacycline (6-methylene oxytetracycline); and doxycycline (.alpha.-6-deoxy-5-hydroxytetracycline). Preferably, the inhibitor of an MMP includes an inhibitor other than an unsaturated fatty acid such as eicosapentaenoic acid.

Other inhibitors include tetracyline derivatives described in U.S. Pat. No. 5,837,696 to Golub et al., which are disclosed to be useful for inhibiting MMP activity in cancer cells. Other classes of MMP inhibitors include the aryl-sulfonyl and related compounds described in U.S. Pat. No. 5,866,587 to de Nanteuil et al. Others include those described by Gowravaram, J. Med. Chem. 38:2570-2581 (1995), which describes the development of a series of hydroxamates that inhibit MMPs and mentions thiols, phosphonates, phosphinates, phosphoramidates and N-carboxy alkyls as known MMP inhibitors. This reference indicates that MMP inhibitors typically may include a moiety that chelates zinc and a peptidic fragment that binds a subset of the specificity pockets of MMPs. Hodgson, Biotechnology 13:554-557 1995 (1995), reviews the clinical status of several MMP inhibitors, including Galardin, Batimastat, and Marimastat. Further MMP inhibitors include butanediamide (Conway et al., J. Exp. Med. 182:449-457 (1995)), TIMPs (Mauch et al., Arch. Dermatol. Res. 287:107-114 (1994)), and retinoids (Fanjul et al., Nature 372:107-111 (1994); Nicholson et al., EMBO Journal 9(13):4443-4454 (1990); and Bailly, C. et al., J. Investig. Derm. 94(1):47-51 (1990)).

Indirect inhibitors may also be used, which include for example, inhibitors of transcription factors such as AP-1 NF-kappa B, and the cascade of factors regulated thereby which are involved in MMP regulation as mentioned in U.S. Pat. No. 5,837,224. Hill, P. A. et al., Biochem. J. 308:167-175 (1995), describes two MMP inhibitors, CT1166 and R0317467, that may regulate MMP transcription factors.

The inhibitor may inhibit multiple types of MMPs, for example, MMP-1 (interstitial collagenase), MMP-2 (72 kD collagenase), MMP-3 (stromelysin), MMP-4 (telopeptidase), MMP-5 (collagen endopeptidase), MMP-6 (acid metalloproteinase), MMP- 7 (uterine metalloproteinase), MMP-8 (neutrophil collagenase), and/or MMP-9 (92 kD collagenase). Inhibitors are preferably selected which preferentially inhibit MMP-25 over the non-type 25 MMPs.

In another embodiment of the method, the inhibitor agent is a nucleic acid or product encoded thereby which is delivered and expressed in the cell by a vector. More specifically, this embodiment of inhibiting the expression of a metalloproteinase includes the steps of administering to the cell a vector comprising a nucleic acid means for inhibiting expression of a MMP-25 polypeptide. Embodiments of this method include those where the nucleic acid means comprises a ribozyme that cleaves an RNA encoding the MMP-25 polypeptide or comprises a molecule that is antisense to a portion of an RNA encoding the MMP-25 polypeptide. In other embodiments of this method, the nucleic acid means is a non-functional variant of a MMP-25 polypeptide. Particularly useful non-functional variants include variants of the amino acid sequence according to SEQ ID NO:2, SEQ ID NO:4 or SEQ ID NO:6; (b) an amino acid sequence having at least 50% identity to the polypeptide of (a) or (b); (c) a polypeptide comprised of a first MMP Zn-binding domain with the proviso that the polypeptide lacks a second MMP Zn-binding domain, and (d) an amino acid sequence encoded by a nucleic acid that hybridizes under conditions of high stringency to (a)-(c).

Method of Modulating Hair Growth

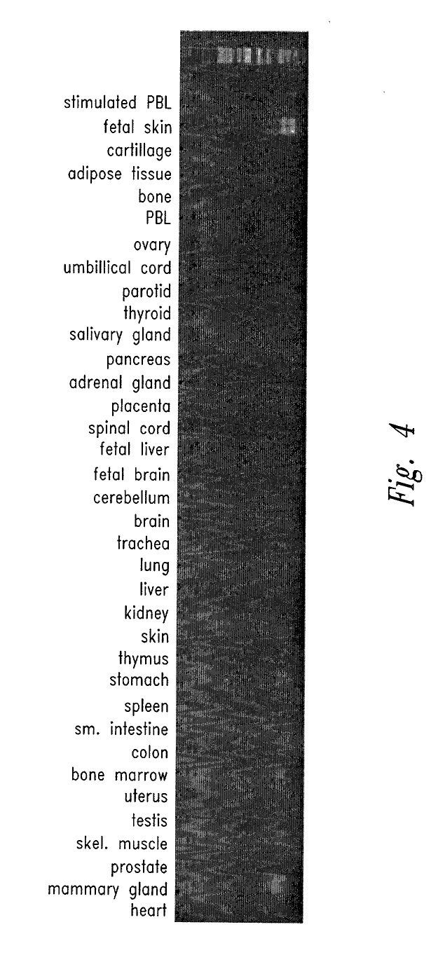

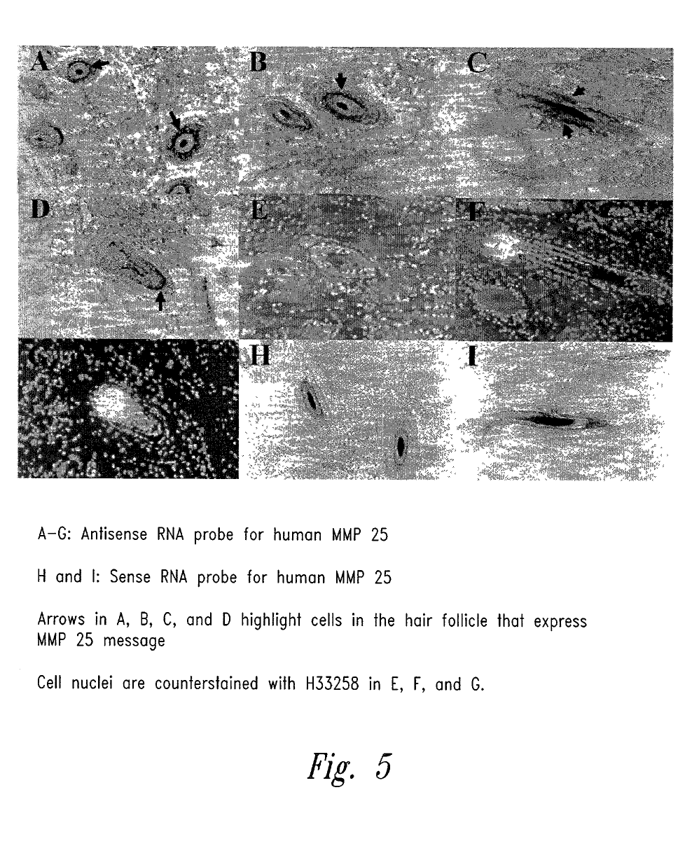

Inhibition of MMP activity generally is known to be a method of inhibiting hair growth as described for example by Styczynski et al. (U.S. Pat. No. 5,962,466). This understanding is based on other MMPs including MMP-1, MMP-3, MMP-4, MMP-5 MMP-6, MMP-7, MMP-8, and more particularly MMP-2 and MMP-9, none of which is known to be preferentially expressed in skin, hair follicles, or especially, active growth cells within follicle tissue. The present invention provides an advantage over these previous methods by identifying a subfamily of MMPs i.e., MMP-25, that is preferentially expressed in cells known to be involved in cell hair growth, namely the basal sheath and particularly the Henle layer of cells of hair follicles as shown in FIGS. 4 and 5.

An improvement in methods of modulating hair growth is provided herein by applying a composition that preferentially inhibits the catalytic activity of MMP-25 to a greater extent than it inhibits the activity of other MMPs, especially other MMPs that may be expressed in cell types of skin tissue. One general method for identification of appropriate inhibitors is described in more detail in Example 5.

In one embodiment of the improved method, a dermatologically acceptable composition comprising a known MMP inhibitor is applied in an amount that inhibits the activity of MMP-25 to a greater extent than it inhibits the activity of other MMPs. Such an inhibitor is preferably incorporated into a topical composition adapted for application to the skin. The amount of inhibitor that preferentially inhibits MMP-25 is determined by assessing the level of reduced MMP catalytic activity against a panel of known MMP enzymes. The zymography procedure described in U.S. Pat. No. 5,962,466 is used to assess relative catalytic activity of the 54 KD MMP-25(l) of the present invention in comparison to the activity of non-type 25 MMPs such as the 72kD MMP-2 and 92 kD MMP-9 present in extracts of skin tissue.

In another practice, a type of inhibitor is selected that preferentially reduces the level of MMP-25 activity over other MMP using a similar assay method. Zymographic separation and activity assessment are conducted as described in Example 4. However, the test samples include any of the wide variety of known MMP inhibitors such those mentioned above, in an amount known to inhibit the activity of MMPs. An inhibitor is selected that preferentially reduces the catalytic activity of a MMP-25 54 kD protein over other MMP activities in the sample.

In either of the above embodiments, once an amount or type of inhibitor is selected, a pharmaceutically acceptable carrier or diluent is formulated to contain test amounts of the selected inhibitor, and applied to the skin of a suitable animal model to determine effective concentration levels. Male intact Golden Syrian hamsters are considered acceptable models for human hair growth as described in more detail in Example 5.

Preferred pharmaceutically acceptable diluents are topical compositions that preferably include a non-toxic, dermatologically acceptable vehicle or carrier which is adapted to be spread on the skin. Examples of suitable vehicles are acetone, alcohols, or a cream, lotion, or gel which can effectively deliver the active compound. One such vehicle is disclosed in PCT/US93/0506A. In addition, a penetration enhancer may be added to the vehicle to further enhance the effectiveness of the formulation.

The concentration of the inhibitor in the composition may be varied over a wide range up to a saturated solution, preferably from 0.1% to 30% by weight or even more. Preferably, an amount of a given inhibitor is selected to preferentially inhibit MMP-25 over non-type 25 MMPs. The effective amounts may range, for example, from 10 to 3000 micrograms or more per square centimeter of skin.

The following examples are offered by way of illustration, and not by way of limitation.

### EXAMPLES

### Example 1

### CLONING OF A LONG MMP-25 CDNA - MMP-25(l)

A first matrix metalloproteinase, herein designated as MMP-25(l), was identified. The polynucleotide encodes a protein comprising the conserved peptide sequences LVAAHELGHXLGLXHSXXXXAXMSSSY (SEQ ID NO:7) and HGDXXPFDGXXXXLAHAFXPGXGXGGDXHPDXDEXWT (SEQ ID NO:8) where X is any amino acid. These conserved peptide sequences represent a consensus for MMP polypeptides as determined by aligning protein sequences of several MMP family members using a multiple sequence alignment program. The consensus sequence is representative of conserved amino acid residues within two separate Zn-binding domains, both of which are ordinarily present on MMPs.

The first MMP sequence identified comprised 833 bp (SEQ ID NO:1). To obtain a full-length cDNA sequence for the novel MMP, a mammary gland cDNA expression library was screened by amplification using RACE reactions with unique sequence primers deduced from the 833 bp sequence in combination with primers that bind to 5′ and 3′ vector sequences adjacent to the ends of cloned inserts. In particular, the vector primer AP1 (Clontech, Palo Alto, Calif.) was used with one of the following primers from the candidate 833 bp sequence to amplify the 5′ sequences:

GSP1: 8563 TGATATCATAATAGATCCTCCATAGGTGCC SEQ ID NO:9

GSP 2: 8564 TTCCTTAGGCAGACCTCCATAGATGGACTGG SEQ ID NO:10

Similarly, the vector primer AP2 (Clontech, Palo Alto, Calif.) was used with one of the following primers from the candidate 833 bp sequence to amplify the 3′ sequences:

GSP3: 7433 CCTAAGGAACCTGCTAAGCCAAAGGAA SEQ ID NO:11

GSP4: 7560 CCGCAGAGAAGTAATGTTCTTTAAA SEQ ID NO:12

Typical RACE reaction conditions were used to amplify cloned sequences, e.g., 35 cycles of a 30 second denaturation followed by a 4 minute extension at between 68 and 72° C. Amplified nucleic acids were isolated and sequenced.

Using the above method, a novel sequence of 1833 bp in length (SEQ ID NO:5) with an open reading frame of 1539 bp (position 12 to 1550 of SEQ ID NO:5) was identified (see, FIG. 2). SEQ ID NO:5 also contained a poly-A tail with a polyadenylation sequence (ATTAAA) located 24 bp upstream (see, FIG. 2), indicative of a true cDNA.

### Example 2

### IDENTIFICATION OF MMP-25 (s)

A second novel metalloproteinase sequence, herein designated MMP-25(s), was also identified by cDNA library screening using RACE reactions as described in EXAMPLE 1. The nucleotide sequence encoding MMP-25(s) is shown in SEQ ID NO:3 and the encoded amino sequence encoded is shown in SEQ ID NO:4. The nucleotide sequence of MMP-25(s) was identical to the sequence for MMP-25(l) except in having a deletion of 129 nucleotides corresponding to 43 amino acids. The deleted sequence in the shorter version of MMP-25 is unique among metalloproteinases: while the encoded protein contains the first Zn-binding domain, it lacks the second Zn/Ca-binding domain typical for other members of the matrix metalloproteinase family as illustrated in FIG. 3.

### Example 3

### TISSUE EXPRESSION PATTERNS OF MMP-25 SEQUENCES

The MMP nucleic acids and polypeptides of the present invention have a unique pattern of tissue expression in human tissue as illustrated in FIG. 4. RT-PCR reactions using reverse transcriptase were performed on RNA samples isolated from a tissue panel from 36 normal tissues. FIG. 4 illustrates that both the long and short variants of MMP-25 were expressed in fetal skin and mammary glands after 35 cycles of amplification, but were poorly detected in other tissues.

The expression in skin tissue is localized in skin follicle cells as illustrated by in situ hybridization results illustrated in FIG. 4. Briefly, fetal skin samples fixed in 4% paraformaldehyde, embedded in paraffin and cut into 5 μm sections were obtained from Biochain Inc. (San Leandro, Calif.) Sections were deparaffinized with xylene and rehydrated using standard procedures. Single-stranded digoxigenin-containing (Roche Molecular Biochemicals (Indianapolis, Ind.) sense and antisense riboprobes were made in vitro using linear templates of MMP-25 DNA and T7 RNA polymerase. Reaction yield and integrity were assessed by gel electrophoresis.

Tissue sections were washed in 10 mM Tris (pH 7.5), 150 mM NaCl for 5 min, followed by a 2 hr blocking step using normal sheep serum (3% final) Sigma, St. Louis Mont.) and 0.035 Triton in 10 mM Tris (pH 7.5) 150 mM NaCl. The slides were incubated with alkaline phosphatase-conjugated anti-DIG antibody (Roche Molecular Biochemicals) at a 1/200 dilution overnight at 4° C. in 10 mM Tris (pH 7.5), 150 mM NaCl supplemented with 1% normal sheep serum. Reference sequential-sections were stained with hemotoxylin and mounted for visualization by light microscopy.

The in situ hybridization results revealed that MMP-25 was expressed in the inner root sheath layer of the hair follicle as shown in FIG. 5. The cell layer within the inner root sheath, the Henle layer, was further defined as a particular cell type for MMPP25 mRNA expression in skin. The particular localization of MMP-25 expression in inner root sheath of hair follicles indicates that control of the expression of the MMP-25 sub-family of metalloproteinases is involved in the regulation of hair growth.

### Example 4

### CHROMOSOMAL LOCATION FOR HUMAN MMP-25

A chromosomal location of MMP-25 was determined using two primers unique to MMP-25 nucleic acids. The primers DMO 7560 (SEQ ID NO:13) and DMO 8563 (SEQ ID NO:14) were used to screen a G3 radiation hybrid panel to map the location of MMP-25. MMP-25 maps to chromosome 11q22, a region where several other MMPs including MMP1, MMP3, MMP7, MMP8, MMP1O, MMP12, and MMP13, have been previously mapped.

### Example 5

### METHOD OF MODULATING HAIR GROWTH

In one practice of an improved method of modulating hair growth, a dermatologically acceptable composition comprising a known MMP inhibitor is applied in an amount that inhibits the activity of MMP-25 to a greater extent than it inhibits the activity of other MMPs. Such an inhibitor is preferably incorporated into a topical composition adapted for application to the skin.

The amount of inhibitor that preferentially inhibits MMP-25 is determined by assessing the level of reduced MMP catalytic activity against a panel of known MMP enzymes. The zymography procedure described in U.S. Pat. No. 5,962,466 is used to assess relative catalytic activity of the 54 KD MMP-25(l) of the present invention in comparison to the activity of the 72kD MMP-2 and 92 kD MMP-9 present in extracts of skin tissue.

Briefly, hair follicles are removed from mammalian skin and homogenized in a non-denaturing buffer, for example a buffer containing 25 mM Tris, H 7.5 and 50 mM sucrose. The samples are prepared for SDS gel electrophoresis and separated on an SDS polyacrylamide gel containing a suitable amount of MMP substrate (e.g., 0.1% gelatin) incorporated therein. The separated proteins are renatured within the gel by incubation with a suitable renaturing buffer such as 2.5% Triton X-100, and renatured in the presence of a buffer containing test amounts of selected MMP inhibitors, for example 0.01 - 10 mM tetracycline, minocyclene, doxycycline, methacycline or 1,10-phenanthroline. The gel is developed in suitable buffer for detecting MMP activity, such as 10 mM Tris base, 40 mM Tris HCl, 200 mM NaCl, 5 mM CaCl₂ and 0.02% Brij 35.

The relative levels of MMP activity and level of inhibition are assessed by detecting the presence and size of “ghost bands” corresponding to the positions of the 54kD 72kD and 92 kD MMP polypeptides after brief staining and destaining of the developed gels with Coomassie blue. Such ghost bands appear relatively transparent against the otherwise relatively opaque background of the stained gelatin due to the proteolytic activity of MMP. Quantitative determinations are made by any of several known means of integration band size such as densitometry. The relative amount of inhibitor that preferentially reduces the activity in the vicinity of the 54kD band relative to the 72kD and 92 kD band is determined.

In another practice, a type of inhibitor is selected that preferentially reduces the level of MMP-25 activity over other MMP using a similar assay method. Zymographic separation and activity assessment are conducted as described above. However, the test samples include any of the wide variety of known MMP inhibitors present in an amount known to inhibit the activity of MMPs. A test inhibitor is selected that preferentially reduces the catalytic activity of MMP-25 54 kD over other MMP activities in the sample. Example test inhibitors include those mentioned above.

Once a relative amount or type of inhibitor is selected as described above, dermatologically acceptable compositions are formulated to contain test amounts of the selected inhibitor and applied to the skin of a suitable animal model to determine desired concentration levels. As disclosed in U.S. Pat. No. 5,962,466, male intact Golden Syrian hamsters are considered acceptable models for human beard hair growth in that they display oval shaped flank organs, one on each side, each about 8 mm in major diameter, which grow thick black and coarse hair similar to human beard hair. These organs produce hair in response to androgens in the hamster.

To evaluate the effectiveness of a composition including an inhibitor of an MMP, the flank organs of each of a group of hamsters are depilated by applying a thioglycolate based chemical depilatory (Surgex). To one organ of each animal a test amount of the vehicle alone once a day is applied, while to the other organ of each animal an equal amount of vehicle containing an inhibitor of a matrix metalloproteinase is applied. After 10 to 15 applications (one application per day for five days a week), the flank organs are shaved and the amount of recovered hair (hair mass) from each is weighed. Percent reduction of hair growth is calculated by subtracting the hair mass (mg) value of the test compound-treated side from the hair mass value of the vehicle-treated side; the delta value obtained is then divided by the hair mass value of the vehicle-treated side, and the resultant number is multiplied by 100.

### Example 6

### ANTISENSE-MEDIATED INACTIVATION OF A MMP-25 PROTEIN

17-nucleotide antisense oligonucleotides are prepared in an overlapping format, in such a way that the 5′ end of the first oligonucleotide overlaps the translation initiating AUG of the MMP-25 transcript, and the 5′ ends of successive oligonucleotides occur in 5 nucleotide increments moving in the 5′ direction (up to 50 nucleotides away), relative to the MMP-25 AUG. Corresponding control oligonucleotides are designed and prepared using equivalent base composition but redistributed in sequence to inhibit any significant hybridization to the coding mRNA. Reagent delivery to the test skin cell system is conducted through cationic lipid delivery (P.L. Feigner, Proc. Natl. Acad. Sci. USA 84:7413, 1987). 2 μg of antisense oligonucleotide is added to 100 μl of reduced serum media (Opti-MEM I reduced serum media; Life Technologies, Gaithersburg Md.) and this is mixed with Lipofectin reagent (6 μl) (Life Technologies, Gaithersburg Md.) in the 100 μl of reduced serum media. These are mixed, allowed to complex for 30 minutes at room temperature and the mixture is added to previously seeded skin cells. These cells are cultured and the mRNA recovered. MMP-25 mRNA is monitored using RT-PCR in conjunction with MMP-25 specific primers such as those used in Example 3 or 4. In addition, separate experimental wells are collected and protein levels characterized through western blot methods using a MMP-25 antibody. The cells are harvested, resuspended in lysis buffer (50 mM Tris pH 7.5, 20 mM NaCl, lmM EDTA, 1% SDS) and the soluble protein collected. This material is applied to 10-20 % gradient denaturing SDS PAGE. The separated proteins are transferred to nitrocellulose and the western blot conducted as above using the antibody reagents described. In parallel, the control oligonucleotides are added to identical cultures and experimental operations are repeated. Decrease in MMP-25 mRNA or protein levels are considered significant if the treatment with the antisense oligonucleotide results in a 25% change in either instance compared to the control scrambled oligonucleotide.

In providing the forgoing description of the invention, citation has been made to several references that will aid in the understanding or practice thereof. All such references are incorporated by reference herein.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. An isolated nucleic acid molecule consisting essentially of a nucleotide sequence selected from the group consisting of:
(a) a nucleotide sequence according to SEQ ID NOs: 1, 3 and 5
(b) a nucleotide sequence having at least 85% identity to the nucleotide sequence according to SEQ ID NOs:1, 3 and 5;
(c) complements of a sequences according to SEQ ID NO: 1, 3 and 5; and
(d) sequences that hybridizes to a sequence according to SEQ ID NO: 1, 3 and 5 under conditions of normal stringency.

2. A polypeptide comprising an amino acid sequence selected from the group consisting of:
(a) an amino acid sequence according to SEQ ID NOs:2, 4 and 6;
(b) an amino acid sequence having at least 90% identity to the amino acid sequence according to SEQ ID NOs:2, 4 and 6;
(c) a nucleotide sequence encoded by a nucleic acid molecule according to claim 1; and
(d) a nucleotide sequence having at least 85% identity to the nucleotide sequence encoded by a nucleic acid molecule according to claim 1; and
(e) an amino acid sequence encoded by a nucleic acid that hybridizes under conditions of normal stringency to the nucleic acid molecule according to claim 1.

3. A method of identifying a nucleic acid molecule encoding all or a part of a metalloproteinase, comprising:
(1) hybridizing a nucleic acid molecule sample to the nucleic acid molecule according to claim 1 and;
(2) identifying a sequence that hybridizes in said nucleic acid sample.

4. The method of claim 3, wherein the step of identifying includes performing a polymerase chain reaction to amplify said hybridizing sequence.

5. An expression vector comprising a nucleic acid molecule according to claim 1 operably linked to an expression control sequence.

6. The vector of claim 5, wherein said vector is selected from the group consisting of plasmid vectors, phage vectors, herpes simplex viral vectors, adenoviral vectors, adenovirus-associated viral vectors and retroviral vectors.

7. A host cell transformed or transfected with an expression vector according to claim 5.

8. A method of producing a polypeptide, comprising culturing a host cell according to claim 7 under conditions allowing for expression of a sequence of the expression vector; and allowing a time sufficient to produce the MMP-25 polypeptide.

9. An antibody that specifically binds to a polypeptide according to claim 2.

10. The antibody according to claim 9 wherein said antibody is a monoclonal antibody.

11. A hybridoma which produces an antibody according to claim 10.

12. A method of identifying a type 25 matrix metalloproteinase, comprising incubating an antibody according to claim 9 with a sample containing a protein; and waiting a time sufficient to permit said antibody to bind type 25 matrix metalloproteinase present in the sample, whereby the binding of the antibody identifies a type 25 matrix metalloproteinase.

13. A fusion protein, comprising at least one polypeptide according to claim 2.

14. A ribozyme that cleaves RNA encoding a polypeptide according to claim 2.

15. An antisense nucleic acid molecule comprising a sequence that is antisense to a portion of a nucleic acid molecule according to claim 1.

16. A method of inhibiting a catalytic activity of a polypeptide according to claim 2, comprising administering an agent to the cell that inhibits a catalytic activity of the said polypeptide, with the proviso that said agent inhibits the catalytic activity of said polypeptide to a greater extent than it inhibits the activity of at least one non-type 25 matrix metalloproteinase.

17. A method of inhibiting the expression of a polypeptide according to claim 2, comprising administering to the cell a vector comprising a nucleic acid molecule which contains a sequence that inhibits expression of a polypeptide according to claim 2.

18. The method of claim 17, wherein said nucleic acid molecule encodes a non-functional variant of a matrix metalloproteinase selected from the group consisting of:
(a) an amino acid sequence according to claim 2;
(b) a polypeptide comprising a first matrix metalloproteinase Zn-binding domain with the proviso that the polypeptide lacks a second matrix metalloproteinase Zn-binding domain; and
(c) an amino acid sequence encoded by a nucleic acid that hybridizes under conditions of high stringency to a nucleic acid molecule according to claim 1.

19. The method of claim 17, wherein said nucleic acid molecule encodes a ribozyme that cleaves a RNA encoding the matrix metalloproteinase -25 polypeptide.

20. The method of claim 17, wherein said nucleic acid molecule contains a sequence that is antisense to a portion of a RNA encoding the matrix metalloproteinase -25 polypeptide.

21. A method of modulating hair growth in a mammal, comprising applying a dermatologically acceptable composition comprising an inhibitor of a matrix metalloproteinase, with the proviso that the applied composition reduces the catalytic activity of a type 25 matrix metalloproteinase to a greater extent than it reduces the catalytic activity of at least one non-type 25 matrix metalloproteinase.

22. A polypeptide according to claim 2, wherein said polypeptide has a first matrix metalloproteinase Zn-binding domain and lacks a second matrix metalloproteinase Zn-binding domain.

23. The polypeptide of claim 22, wherein said polypeptide exhibits a catalytic activity of a matrix metalloproteinase.

24. The polypeptide of claim 22, wherein said polypeptide lacks a catalytic activity of a matrix metalloproteinase.
